(19) United States
(12) Patent Application Publication
(10) Pub. No.: US 2010/0312139 A1
(43) Pub. Date: Dec. 9, 2010

(54) Method for Identifying Mammals at Risk for Elevated intracranial Pressure

(75) Inventors: Pramod K. DASH, Pearland, TX (US); John B. REDELL, Houston, TX (US); Georgene Hergenroeder, Houston, TX (US); Anthony N. MOORE, Sugar Land, TX (US)

Correspondence Address: CONLEY ROSE, P.C.;David A. Rose, P. O. BOX 3267, HOUSTON, TX, 77253-3267, US

(73) Assignee: BOARD OF REGENTS OF THE UNIVERSITY OF TEXAS SYSTEM, Austin, TX (US)

(21) Appl. No.: 12/793,820

(22) Filed: Jun. 4, 2010

Related U.S. Application Data
(60) Provisional application No. 61/184,137, filed on Jun. 4, 2009.

(51) Int. Cl. A61B 5/00; G01N 33/20; G01N 33/68; C12Q 1/00; G01N 33/543; G01N 33/53

(52) U.S. Cl. 600/561; 436/76; 436/80; 435/4; 436/518; 435/7.8

(57) ABSTRACT

Disclosed are methods of diagnosing, screening and treating individuals at risk of elevated intracranial pressure based on detecting a transient decrease in serum ceruloplasmin and/or serum copper levels in the individual soon after occurrence of a head injury. This early drop in ceruloplasmin or copper level indicates an impending, potentially life threatening, elevation in the individual's intracranial pressure. Also disclosed are diagnostic applications and processes for identifying and producing diagnostic and prognostic assays useful for predicting elevated intracranial pressure, and test kits for performing such tests.

### CROSS-REFERENCE TO RELATED APPLICATIONS

This application claims the benefit under 35 U.S.C. §119(e) of U.S. Provisional Patent Application No. 61/184,137 filed Jun. 4, 2009, the disclosure of which is hereby incorporated herein by reference.

### STATEMENT REGARDING FEDERALLY SPONSORED RESEARCH OR DEVELOPMENT

Not applicable.

### BACKGROUND

1. Technical Field

The disclosure generally relates to the diagnosis and treatment of individuals at risk of developing elevated intracranial pressure (ICP) also known as intracranial hypertension. More particularly, the disclosure pertains to diagnostic and treatment methods that include detecting a decrease in the level of ceruloplasmin and/or copper to identify individuals who will develop elevated ICP, and to kits for performing such methods.

2. Description of Related Art

Traumatic brain injury (TBI) is the result of a blow or jolt to the head or a penetrating head injury that disrupts the normal function of the brain. Not all such blows to the head will result in a TBI. TBI can vary in severity from relatively mild (i.e., concussion) to severe. Mild TBI may manifest itself as a brief (seconds or minutes) change in consciousness and/or mental status, for example, trouble with attention, concentration, thought processes or memory, changes in sleep patterns, behavior or mood. Mild TBI may include loss of consciousness, post-traumatic amnesia, altered mental status and/or focal neurologic deficits and seizure. Mild TBI has also been associated with headache, confusion, lightheadedness, dizziness, blurred vision or eye strain, a ringing in the ears, a bad taste in the mouth, and fatigue or lethargy.

Moderate or severe TBI may be associated with amnesia or loss of consciousness for an extended period of time and many of the same symptoms as mild TBI, but the headache persists or worsens, vomiting or nausea may be repeated, there may be convulsions or seizures, dilation of one or both pupils of the eyes, slurred speech, weakness or numbness in the extremities, loss of coordination, and increased confusion, restlessness, or agitation or an inability to awaken from sleep.

According to the Centers for Disease Control (CDC: Langlois J A, Rutland-Brown W, Thomas K E. Traumatic brain injury in the United States: emergency department visits, hospitalizations, and deaths. Atlanta (GA): Centers for Disease Control and Prevention, National Center for Injury Prevention and Control; 2004). TBI contributes to a substantial number of deaths and cases of permanent disability. Each year in the U.S. a total of 1.4 million people sustain TBI, of these 1.1 million are treated and released from an emergency rooms, 235,000 are hospitalized and 50,000 will die. In children ages 0 to 14 years, TBI results in an estimated: 435,000 emergency room visits, 37,000 hospitalizations and 2,685 deaths. The CDC estimates that as a result of a TBI, at least 5.3 million Americans, or approximately 2% of the U.S. population, currently have a long-term or lifelong need for help to perform activities of daily living. The leading causes of TBI are: falls, motor vehicle, bicycle and pedestrian accidents, sports related collisions, and assaults such as but not limited to shaken baby syndrome or head wounds. In the year 2000, direct medical costs and indirect costs such as lost productivity of TBI totaled an estimated $60 billion (Finkelstein E, Corso P, Miller T and associates. The Incidence and Economic Burden of Injuries in the United States. New York (NY): Oxford University Press; 2006).

In addition, while members of the military have always been at risk for TBI, the frequent deployment of improvised explosive devices (IEDs) in the current wars in Iraq (Operation Iraqi Freedom) and Afghanistan (Operation Enduring Freedom) have dramatically increased these risks. As reported in a recent RAND Corporation study (Tanielian T and Jaycox L H, eds., “Invisible Wounds of War: Psychological and Cognitive Injuries, Their Consequences, and Services to Assist Recovery”, Santa Monica, Calif.: RAND Corporation, MG-720-CCF, 2008, 492 pp) a survey of returning veterans revealed that 19.5% of the respondents had suffered TBI, If this data is generally applicable to all personnel deployed in these conflicts, this translates to as many as 320,000 cases of TBI.

Elevated intracranial pressure (ICP) is a prominent secondary pathology after traumatic brain injury (TBI), and is a major contributor to morbidity and mortality. Identification of prognostic biomarkers for identifying those patients at risk for developing elevated ICP would greatly aid in the clinical management of these patients (Shore, P. M., Berger, R. P., Varma, S., et al., 2007. Cerebrospinal fluid biomarkers versus Glasgow coma scale (GCS) and Glasgow outcome scale in pediatric traumatic brain injury: the role of young age and inflicted injury. J. Neurotrauma 24, 75-86, and Wang, K. K., Ottens, A. K., Liu, M. C., et al., 2005. Proteomic identification of biomarkers of traumatic brain injury. Expert. Rev. Proteomics. 2, 603-614).

However, among those patients with severe TBI, initial GCS was found to not be significantly different between patients with normal ICP and those who develop elevated ICP, nor did it have a high degree of sensitivity (33%) in stratifying these patients (Zemlan, F. P., Jauch, E. C., Mulchahey, J. J., et al. 2002. C-tau biomarker of neuronal damage in severe brain injured patients: association with elevated intracranial pressure and clinical outcome. Brain Res. 947, 131-139).

In a preliminary clinical study, Zemlan et al. 2002 (supra), have reported that the initial CSF levels of cleaved tau (c-tau) can be used as a biomarker for predicting which patients are likely to develop elevated ICP. This study found that the levels of CSF c-tau measured on day 1 after injury was increased in patients who subsequently developed elevated ICP (>30 mm Hg) compared to those whose ICP remained <30 mm Hg. While CSF is an excellent source for biomarker discovery, its usefulness in the diagnostic process remains limited. Using serum samples from severely injured TBI patients, Petzold et al., 2002 (Petzold, A., Green, A. J., Keir, G., et al., 2002. Role of serum S100β as an early predictor of elevated intracranial pressure and mortality in brain injury: a pilot study. Crit Care Med. 30, 2705-2710), reported that the levels of S100β recorded during the first 24 hr of injury were significantly higher in patients who develop elevated ICP than those whose ICP remained in the normal range. However, recent reports have challenged the utility of S100β as a diagnostic marker of brain damage, necessitating the continued search for prognostic biomarkers of elevated ICP.

Currently, there are no clinically proven methods for predicting which TBI patients will develop elevated ICP. Therefore there is a clear and outstanding need for a method of identifying those at high risk for developing elevated ICP (also known as intracranial hypertension), as a result of TBI, stroke and other brain injury or related disorders.

### BRIEF SUMMARY

In accordance with certain embodiments of the invention, a process for identifying a patient at risk for elevated intracranial pressure is provided which comprises analyzing a test sample, e.g., a body fluid such as blood serum, obtained from a patient for ceruloplasmin levels, copper levels or both, and determining that serum ceruloplasmin levels, serum copper levels or both levels are decreased below the normal range on the patient who has experienced a possible brain injury.

In accordance with some embodiments of the invention, a process for identifying a patient at risk for elevated intracranial pressure in provided, wherein the patient has experienced a precipitating event that includes brain injury or potential brain injury. The process comprises measuring a ceruloplasmin level, copper level, or both ceruloplasmin and copper levels in a test sample of body fluid obtained from the patient, wherein the test sample is obtained within 36 hours after the precipitating event; and determining from the measurement that the serum ceruloplasmin level, serum copper level, or both levels, are below the respective normal ranges, wherein a subnormal level of either serum ceruloplasmin or serum copper, or both, indicates increased risk for elevated intracranial pressure. In some embodiments the body fluid comprises serum. In some embodiments the body fluid is plasma, blood, urine, cerebral spinal fluid, urine or saliva, and the step of determining includes correlating the measured levels to a serum ceruloplasmin level, a serum copper level, or both.

In accordance with certain embodiments of the invention, a method is provided for screening a population of individuals for increased risk of elevated intracranial pressure, wherein the individuals have experienced a precipitating event that may include or cause brain injury, the method comprising: (a) measuring ceruloplasmin level or copper level, or both ceruloplasmin and copper levels, in a body fluid sample obtained from each said individual within 36 hours after the precipitating event, wherein a subnormal level of either serum ceruloplasmin or serum copper, or subnormal serum levels of both ceruloplasmin and copper, is/are a positive test result indicative of increased risk of elevated ICP. In some embodiments the body fluid is serum. In some embodiments, the body fluid is plasma, blood, urine, cerebral spinal fluid, urine or saliva, and the step of measuring includes correlating the measured levels to a serum ceruloplasmin level, a serum copper level, or to both levels. The method further comprises (b) monitoring at least the individuals with a positive test result at an increased frequency and level of intensity (e.g., observation in an intensive care unit where the personnel are trained to respond to subtle changes and the equipment is available to monitor patients and respond immediately.) For example, in some instances, increased intensity of monitoring includes dedication of additional resources, whether that be manpower or equipment, to ensure that patients at risk for developing elevated ICP are able to be treated in a time-efficient manner. This may involve, but is not limited to, the scheduling of surgical interventions, transport of the patient, imaging, placement of monitoring systems, and other appropriate steps. In some cases, a more intense level of monitoring includes the use of aggressive medical therapies/interventions. In some embodiments, with respect to at least the individuals with positive test results, an above-described method includes (c) taking action to ensure that neurosurgical resources and personnel are in attendance prior to onset of elevated ICP in the individual. In certain embodiments, in which measurement of copper levels are desired, the test sample is obtained within 24 hours after the precipitating event.

Also provided in accordance with certain embodiments is a method of treating an individual who has experienced a precipitating event that includes or may cause brain injury. This method comprises (a) measuring ceruloplasmin level or copper level, or both ceruloplasmin and copper levels, in a body fluid sample obtained from the individual within 36 hours after the precipitating event. Ceruloplasmin is preferably measured within 36 hours and copper is preferably measured within 24 hours after the precipitating event. A subnormal level of either serum ceruloplasmin or serum copper, or subnormal serum levels of both ceruloplasmin and copper, is/are a positive test result indicative of increased risk of elevated ICP. The method further includes (b) if the test result is positive, monitoring the individual at an increased frequency and level of intensity; and (c) if the test result is positive, taking action to ensure that the neurosurgical resources and personnel are in attendance prior to onset of ICP in the individual.

In some embodiments of an above-described method the increased level of intensity includes monitoring at least some of the following parameters and/or symptoms in the patient: level of consciousness, verbal responsiveness and appropriateness of the responses, pupillary response, vision, visual changes, motor function, headache, vomiting, blood pressure, change in blood pressure, pulse rate, change in pulse, respiratory pattern, change in respiratory pattern, body temperature and change in temperature. In certain embodiments, at least the following symptoms are documented: headache, vomiting, confusion, seizures, amnesia, paralysis and spasticity.

In some embodiments of an above-described method, step (c) includes, prior to onset of elevated ICP, transporting the individual or individuals with positive test results to a medical facility with the neurological resources and personnel to treat the individual. In some embodiments an above-described method includes step (d) administering to at least the individuals having a positive test result a treatment to deter or alleviate elevated ICP.

In various embodiments of the above-described methods or processes, the test sample is obtained within 36 hrs, 35 hrs., 34 hrs., 33 hrs., 32 hrs., 31 hrs., 30 hrs., 29 hrs., 28 hrs., 27 hrs., 26 hrs., 25 hrs., 24 hrs., 23 hrs., 22 hrs., 21 hrs., 20 hrs., 19 hrs. 18 hrs., 17 hrs., 16 hrs., 15 hrs., 14 hrs., 13 hrs., 12 hrs., 11 hrs., 10 hrs., 9 hrs., 8 hrs., 7 hrs., 6 hrs., 5 hrs., 4 hrs., 3 hrs., 2 hrs., 1 hrs., 45 min., or 30 min. of the precipitating event that includes a brain injury or potential brain injury. Unbound serum ceruloplasmin generally has a half-life of about 5 hrs. in the adult human. Therefore, in some applications the body fluid sample is taken between 2-36 hrs. after the precipitating event, as a significant drop in ceruloplasmin level might not occur earlier than about 2 hrs. after a precipitating event.

In accordance with still another embodiment of the invention, a test kit for identifying an individual at increased risk of elevated intracranial pressure is provided which includes: (a) a first set of components for measuring copper levels in a body fluid sample; and (b) literature containing instructions (i) for obtaining a body fluid sample from the individual within 36 hours after a precipitating event that may include brain injury, or may lead to brain injury, in the individual; (ii) for using the first set of components to measure copper level in the body fluid sample; and (iii) for interpreting a resulting copper level, wherein a subnormal level of serum copper indicates increased risk of elevated ICP. In some embodiments, the kit additionally includes (c) a second set of components for measuring ceruloplasmin levels in a body fluid sample, and the included literature further comprises instructions (iv) for using the second set of components to measure ceruloplasmin level in the body fluid sample; and instructions (v) for interpreting a resulting ceruloplasmin level, wherein a subnormal level of serum ceruloplasmin indicates increased risk of developing elevated ICP. These and other embodiments, and various features and advantages will be apparent in the description and drawings.

### BRIEF DESCRIPTION OF THE DRAWINGS

FIG. 1 shows the ceruloplasmin levels in pooled serum samples obtained at either 2 days or 3 days post-TBI determined from an isobaric tag for relative and absolute quantitation (iTRAQ)-labeled ceruloplasmin peptide fragment by MS/MS sequence spectrum. FIG. 1A is an expanded m/z scale corresponding to the region containing an iTRAQ-tagged ceruloplasmin peptide indicated by an asterisk. FIG. 1B shows the MS/MS sequence spectrum for the peptide from FIG. 1A mapping to the ceruloplasmin protein showing the y- and b-series ions of the respective collision-induced fragments. The deduced amino acid sequence is shown above the spectrum. FIG. 1C shows a representative spectrum indicating the intensity of the respective isobaric tag signals from healthy volunteer (115 Da), 2 day TBI patient (116 Da) and 3 day TBI patient (117 Da) samples. The time points of the pooled samples were 41.5+/−4.9 h and 66.3+/−6.6 h.

FIG. 2 shows serum ceruloplasmin levels were increased 3 days post-TBI. FIG. 2A provides demographic information for the subjects used for ELISA analysis. FIG. 2 is a standard curve for the ELISA assay using purified ceruloplasmin protein. FIG. 2C shows serum concentration of ceruloplasmin in samples obtained from healthy volunteers (HV, n=20), and brain-injury patients (n=20) on day 2 (41.5±4.5 hr) and day 3 (66.3±6.6 hr) post-injury. Data is presented as the mean±SEM. *, p<0.05 compared to HV by one-way ANOVA. FIG. 2D is a picture of a representative Western immunoblot using ceruloplasmin antibody from the ELISA kit, described in Example 1.4 (GenWay, San Diego, Calif.) to detect purified ceruloplasmin, and ceruloplasmin in pooled serum samples from healthy volunteers and the TBI patients taken 3 days post-injury.

FIG. 3 shows serum concentration of ceruloplasmin in subject populations, as determined by ELISA, stratified: by ICP<20 mm Hg versus ICP>25 mm Hg) in FIG. 3A; by injury severity score (ISS)<29 versus ISS≧29, in FIG. 3B; by head trauma versus polytrauma, in FIG. 3C; by male versus female, in FIG. 3D; by age<26 years versus age>26 yrs, in FIG. 3E; and by hispanic versus non-hispanic, in FIG. 3F. All data is presented as mean±SEM. [symbol] , interaction by two-way ANOVA. *, p<0.05 by post-hoc analysis.

FIG. 4 demonstrates that ceruloplasmin levels predict subsequent ICP elevations. FIG. 4A is a scatter plot showing the relationship between time of sample withdrawal and ceruloplasmin levels (left). Serum concentration of ceruloplasmin as a function of time plotted as 12 hr bins from injury in patient groups whose ICP remained under 20 mm Hg and those whose maximum ICP was ≧25 mm Hg (right). FIG. 4B is a dot histogram for the concentrations of ceruloplasmin measured in individual patients during the first 24 hr of injury. FIG. 4C is a ROC curve yielding an AUC value of 0.87 indicating that the serum level of this protein is a very good predictor of ICP status. Data is presented as mean±SEM. [symbol] , group main effect by two-way ANOVA. *, p<0.05 by post-hoc analysis.

FIG. 5 demonstrates that serum copper levels predict subsequent ICP elevations. FIG. 5A shows a standard curve for total copper obtained using CuSO₄ as the standard. FIG. 5B shows the concentration of total serum copper in human volunteers, in TBI patients with ICP<20 mm Hg, and in TBI patients with ICP≧25 mm Hg. FIG. 5C shows a dot histogram for concentration of copper in individual patients during the first 24 hr of injury. FIG. 5D shows a ROC curve for total serum copper levels indicating that copper is a very good prognostic marker for elevated ICP. Data is presented as mean±SEM. [symbol] , group main effect by two-way ANOVA*, p<0.05 by post-hoc analysis.

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The term “increased intracranial pressure” or “ICP,” as used herein, unless otherwise indicated, generally refers to pressure above about 20 mm Hg and is usually considered a clinical treatment threshold for ICP in human adults. Normal ICP in human adults is generally considered to be in the range of 0-15 mm Hg.

As used herein, and unless otherwise indicated, the terms “treat”, “treating”, and “treatment” contemplate an action that occurs while a patient is suffering from elevated ICP that reduces the severity of one or more symptoms or effects of elevated ICP, or a related disease or disorder. Where the context allows, the terms “treat”, “treating”, and “treatment” also refers to actions taken toward ensuring that individuals at increased risk of elevated ICP are able to receive appropriate neurosurgical or other medical intervention prior to onset of elevated ICP.

As used herein, and unless otherwise indicated, the terms “prevent”, “preventing”, and “prevention” contemplates a prophylactic action that prolongs the onset of, and/or inhibits or reduces the severity of, elevated ICP. “Prevention” contemplates an action that occurs before a patient begins to suffer from elevated ICP, that prolongs the onset of, and/or inhibits or reduces the severity of, elevated ICP.

As used herein, and unless otherwise indicated, the terms “manage”, “managing”, and “management” encompass preventing, delaying, or reducing the severity of elevated ICP in a patient who is already suffering from such a disease or condition. “Manage”, “managing”, and “management” also encompass preventing, delaying, or reducing the severity of a recurrence of elevated ICP in a patient who has already suffered from such a disease or condition. This may include modulating the threshold, development, and/or duration of the elevated ICP or changing how a patient responds to the elevated ICP.

As used herein, and unless otherwise specified, a “therapeutically effective amount” of a compound is an amount sufficient to provide any therapeutic benefit in the treatment or management of elevated ICP or to delay or minimize one or more symptoms associated with elevated ICP. A therapeutically effective amount of a compound means an amount of the compound, alone or in combination with one or more other therapies and/or therapeutic agents, that provides any therapeutic benefit in the treatment or management of elevated ICP, or related diseases or disorders. The term “therapeutically effective amount” can encompass an amount that alleviates elevated ICP, improves or reduces elevated ICP, improves overall therapy, or enhances the therapeutic efficacy of another therapeutic agent.

As used herein, and unless otherwise specified, a “prophylactically effective amount” of a compound is an amount sufficient to prevent or delay the onset of elevated ICP, or one or more symptoms associated with elevated ICP or prevent or delay its recurrence. A prophylactically effective amount of a compound means an amount of the compound, alone or in combination with one or more other treatment and/or prophylactic agent that provides a prophylactic benefit in the prevention of elevated ICP. The term “prophylactically effective amount” can encompass an amount that prevents elevated ICP, improves overall prophylaxis, or enhances the prophylactic efficacy of another prophylactic agent.

### Overview

It was discovered that the level of the mammalian protein ceruloplasmin or caeruloplasmin (IUBMB Enzyme Nomenclature: EC 1.16.3.1, which is also known or referred to as: caeruloplasmin, ferroxidase I; iron (II): oxygen oxidoreductase; ferro: O₂ oxidoreductase; ferroxidase, and iron II: oxygen oxidoreductase), and the level of copper decrease in the serum of an individual following brain injury. These decreases occur soon after the injury but before dangerous rises in ICP occur. This information can be used to accurately predict which individuals are at increased risk for developing potentially life threatening elevations in ICP. This association of decreased serum ceruloplasmin and copper levels with an impending rise in ICP was previously unknown.

Elevated ICP can occur when the brain is injured, swelling occurs and fluids accumulate within the brain space. It is normal for bodily injuries to cause swelling and disruptions in fluid balance, but when an injury occurs inside the skull-encased brain, there is no place for swollen tissues to expand and no adjoining tissues to absorb excess fluid. Elevated ICP levels occur when an increase in the volume of one or more components of the brain cannot be offset by a reduction or displacement in one of the other components. Elevated ICP can result as a consequence of traumatic brain injury (TBI) or other related disorders, such as, but not limited to, brain injury, head trauma, polytrauma, stroke, meningitis, encephalitis, hydrocephalus, spinal cord injury, blocked shunts, tumors, for example those of the central nervous system (CNS) which increase ICP, for example those that cause impingement or occlusion.

Elevated ICP is a prominent secondary pathology after TBI, and is a major contributor to morbidity and mortality of TBI and other related disorders such as, but not limited to, brain injury, head trauma, polytrauma, stroke, meningitis, encephalitis, hydrocephalus, spinal cord injury, blocked shunts, tumors, for example those of the central nervous system (CNS) which elevated ICP, for example those that cause impingement or occlusion. Currently, there are no clinically proven methods for predicting which patients at risk will develop elevated ICP.

Elevated ICP may result from the initial brain trauma or from secondary injury to the brain. In adults, normal ICP is considered 0-15 mm Hg, current recommended practice is to initiate treatment to lower ICP when ICP levels are between 20-25 mm Hg, although it should be noted that in some cases patients' brain tissue structures can herniate with ICP levels below 20, and abnormal pupil responses have been noted at ICP levels as low as 18 mm Hg.

Elevated ICP, a major contributor to the mortality and morbidity associated with brain trauma, typically develops slowly after injury, peaking on the second or third day after injury. Thus the peak incidence of elevated ICP after TBI, for example, is thought to occur in the first 72 hours post injury although others have noted that clinical deterioration from associated brain swelling most commonly occurring 48-96 hrs post-ictus. As many as 66% of the TBI patients develop increased ICP within the first 24 hr and clinical deterioration from associated brain swelling most commonly occurring 48-96 hrs post-ictus. Of course, not all elevations are deleterious. An increase from 1 to 2 mm Hg is usually considered insignificant and harmless For example, sneezing and coughing increases ICP, but they are well tolerated in the healthy brain. Furthermore, an increase from 6 to 12 mm Hg may represent an increase, but damage caused by this increase is not necessarily assumed. In the types and levels of ICP usually associated with the TBI, stroke, and other ICP-precipitating conditions that are encountered in a clinical setting, the clinical treatment threshold is generally ICPs above about 20 mm Hg.

Typically, if a patient is suspected of developing elevated ICP, he may be subject to the insertion of a direct ICP monitoring device (i.e., ventricular catheter, subarachnoid bolt, subdural catheter, fiberoptic or pneumatic transducer, or multimodal monitors utilizing fiberoptic pressure sensors and strain gauge transducers), typically into the lateral ventricle, subarachnoid space, or epidural space. To measure a patient's ICP directly, medical personnel generally use a probe or catheter. The instrument is inserted through the skull, using, for example, a bolt (e.g., a Camino bolt), to the subarachnoid level and is connected to a monitor that registers the patient's ICP. There are, however, several drawbacks to direct monitoring of patient ICP. It is an invasive procedure that requires that a hole be drilled in the patient's skull, a procedure that is best done by highly trained personnel, and like all invasive procedures it carries with it the risk of infection, and it also carries the risks associated with surgical procedures, including bleeding and post-operative infection, as well as risks associated with damage to nearby organs. Even greater risks are associated with nearby vital organs such as the brain. Severe elevations in ICP are particularly dangerous because, in addition to creating a significant risk for cerebral ischemia, uncontrolled ICP may result in the herniation of brain structures. Elevations in ICP may also require a ventriculostomy, which is an invasive procedure that drains cerebrospinal fluid (CSF) from the brain to bring the ICP down. Drugs such as mannitol, barbiturates and other suitable drugs are sometimes used to decrease ICP.

In addition, direct monitoring of patient ICP requires trained personnel to constantly monitor the patients ICP as well as to react to changes. In many cases, the reaction to severely elevated ICP requires a neurosurgeon and a neurosurgical team to be readily available. Such methods of ICP monitoring are not always available, are not always utilized, nor are all facilities adequately equipped to deal with sudden rises in ICP. For example, in an outlying hospital or medical facility such as that in a rural setting, highly trained neurosurgical teams may not be readily available. The use of an easily obtained early marker of impending ICP elevation, as disclosed herein, would allow advanced warning of imminent life threatening ICP levels. This would provide time limited neurosurgical resources and personnel to be summoned and/or it would provide potentially life-saving time to transport the patient, for example by helicopter (such as “Life Flight”), to a higher level trauma facility where neurosurgeons and neurosurgical teams and other more specialized resources are more readily available. Thus, in certain embodiments a treatment method includes facilitating a more effective allocation of limited medical resources.

In certain embodiments a treatment method that includes advance detection of a decreased level of ceruloplasmin and/or copper includes transforming a trauma patient's status from being one who is potentially or theoretically at risk for developing elevated ICP to one that is clearly at high risk of developing elevated ICP. This transforms the patient's evaluation from a subjective to objective assessment. As a high risk patient, he is a candidate for more invasive intracranial monitoring techniques, including, but not limited to, the insertion of a bolt (e.g., a Camino bolt), a ventriculostomy, which both monitors and allows for CSF drainage and may very likely require additional specialized intervention, such as a decompressive craniectomy.

Patients with mild to moderate trauma injuries may receive skull and neck X-rays to check for bone fractures or spinal cord compression or instability. For moderate to severe cases, the imaging test is a computed tomography (CT) scan. Imaging tests help in determining the diagnosis and prognosis of a trauma patient. One common cause of elevated ICP is TBI which can result from a blow or jolt to the head or from a penetrating head injury that disrupts the normal function of the brain. Not all head blows results in a TBI and when it occurs TBI can vary in severity from relatively mild to severe.

Mild TBI can manifest itself as a brief change in consciousness and/or mental status, lasting only seconds to minutes, such as for example, trouble maintaining attention, concentration, thought processes or memory, changes in sleep patterns, behavior or mood, amnesia of the event, or brief loss of consciousness. Mild TBI has also been associated with headache, confusion, lightheadedness, dizziness, blurred vision or eye strain, a ringing in the ears, a bad taste in the mouth, and fatigue or lethargy. Moderate or severe TBI can be associated with amnesia or loss of consciousness for an extended period of time and many of the same symptoms as mild TBI, but the headache persists or worsens, vomiting or nausea is repeated, there are convulsions or seizures, dilation of one or both pupils of the eyes, slurred speech, weakness or numbness in the extremities, loss of coordination, and increased confusion, restlessness, or agitation or an inability to awaken from sleep.

Anyone with signs of moderate or severe TBI should receive medical attention as soon as possible. Because little can be done to reverse the immediate brain damage caused by the initial trauma, medical personnel try to stabilize an individual with TBI and focus on preventing further injury by minimizing secondary brain injury, such as can be caused by elevated ICP. Therefore, rapid and noninvasive identification of patients who are at the highest risk for deleteriously elevated ICP is critical information and is provided by many embodiments of the diagnostic and treatment methods described herein.

The severity of head injuries is classified clinically by the Glasgow Coma Scale (GCS). A GCS score of 13 to 15 is classified as a mild head injury, a score of 9 to 12 as moderate, and a score of <8 as severe. The relationship between initial GCS and clinical outcome has been well established. However, it has been determined that no significant difference in initial GCS was observed among those patients with severe TBI, between those who went on to develop elevated ICP and those who did not.

Primary concerns for minimizing secondary brain injury include insuring proper oxygen supply to the brain and the rest of the body, maintaining adequate blood flow, and controlling blood pressure. Secondary brain injury is any subsequent injury to the brain after the initial insult. Secondary brain injury can result from systemic hypotension, hypoxia, elevated ICP or as the biochemical result of a series of physiologic changes initiated by the original trauma.

### Advance Detection of Impending Elevation of ICP.

It was discovered that, in at-risk patients, those who go on to develop elevated ICP have a transient but significant reduction in ceruloplasmin and copper levels below the normal range shortly after injury but well before elevated ICP occurs or peaks. For example, the level of ceruloplasmin and/or copper in the patient's body fluid sample is/are determined to be reduced within the first 36 hrs after the injury or other precipitating event. An example of a suitable body fluid for testing is blood serum. In some embodiments, panels of copper and ceruloplasmin levels in another body fluid (e.g., plasma, blood, cerebral spinal fluid, urine and saliva) are established in a manner similar to that described herein for serum, and are correlated to serum levels. In this way, copper and/or ceruloplasmin levels in a body fluid other than serum are used as surrogate markers indicative of serum levels, wherein a subnormal level of either serum ceruloplasmin or serum copper, or both, indicates increased risk for elevated intracranial pressure.

By screening at-risk patients for an early post-injury drop in ceruloplasmin and/or copper levels, it can be better predicted which of those individuals will go on to develop elevated ICP. In certain embodiments, a noninvasive, rapid and inexpensive method and testing kit is provided to predict which patients will experience elevated ICP. This knowledge provides an early warning system that facilitates the more rapid and appropriate deployment of resources, allowing time to transport a patient to a tertiary care center or time to assemble a neurosurgeon and surgical team. In many embodiments, this method advantageously provides the time to initiate early intervention so that deleterious elevation of ICP and related damage is limited or avoided altogether.

Drug and alcohol consumption can often cloud the evaluation of trauma patients, either making patients appear more severely injured or, sometimes worse, causing the medical staff to miss a patient who, while under the influence, has also suffered a brain injury or stroke and is at high risk for potentially fatal elevations in ICP, for example, once they are released from the hospital and have returned home. Therefore, in some instances, a testing method facilitates the differentiation of those trauma patients whose symptoms appear to be exacerbated or are masked by drug or alcohol use from those trauma patients who are truly at risk for elevated ICP.

Additionally, in instances of undisclosed brain injury, as may be the case in shaken baby syndrome, where unreported trauma, non-specific symptoms and unremarkable physical exam may be indistinguishable from other childhood illnesses (e.g., irritability, vomiting), this method of differentiation may facilitate diagnosis and therefore alert providers to the need for brain imaging (e.g., head CT or DTI) and implementation of abuse prevention measures or additional treatments.

In some embodiments, a disclosed method facilitates identification of individual mammals that are at increased risk of potentially fatal elevated ICP based on the discovery that reduced ceruloplasmin and copper levels predict increased ICP in human TBI patients.

In some embodiments, a patient at risk for elevated ICP is identified according to a method that includes analyzing a test sample obtained from the patient to determine the level of ceruloplasmin and/or copper in the sample. When it is determined that the patient's serum ceruloplasmin and/or copper levels fall below the normal range, that patient is identified as being at high risk for deleteriously elevated ICP. In certain embodiments, the test sample obtained is a blood serum sample from the patient. In various other embodiments the test sample is a body fluid comprising plasma, blood, urine, cerebral spinal fluid (CSF), urine or saliva.

In certain embodiments, the patient of interest has suffered a possible brain injury. In other embodiments, that possible brain injury is due to traumatic brain injury, head trauma, polytrauma, stroke, meningitis, encephalitis, hydrocephalus, spinal cord injury, blocked shunts, and certain tumors for example those of the central nervous system (CNS) which increase ICP, for example those that cause impingement or occlusion.

In certain embodiments, the patient's ceruloplasmin levels are determined using an immunoassay. In one embodiment that immunoassay comprises the use of an enzyme-linked immunoassay. In another, that immunoassay comprises the use of a radioimmunoassay. In still another embodiment that immunoassay comprises the use of immunonephelometry. Another embodiment is one wherein the immunoassay comprises the use of immunochromatography and in another embodiment that use of immunochromatography comprises a lateral flow test strip or any other known assay of ceruloplasmin (including but not limited to native or modified proteins, peptides and/or metabolic break down products of ceruloplasmin (e.g., ceruloplasmin peptides or modified ceruloplasmin) that is applicable to use with a body fluid.

The determination of copper levels in a patient's body fluid comprises analysis using a colorimetric assay. In other embodiments the determination of copper levels in the patient's body fluid comprises analysis using an assay based on either spectrophotometry or atomic absorption spectroscopy or flame atomic absorption spectroscopy or inductively coupled plasma/mass spectrometry or any other known assay of copper that is applicable to use with a body fluid.

In some embodiments, it is determined whether an individual is a candidate for an invasive intracranial pressure monitoring technique such as those requiring the insertion of an intracranial pressure monitoring device. This method comprises analyzing a test sample of body fluid obtained from the individual after a possible brain injury for ceruloplasmin level, copper level, or both, in the sample and determining whether the serum ceruloplasmin level, the serum copper level, or both, are decreased below the normal range. A subnormal value of either serum ceruloplasmin or serum copper, or both, is considered a positive result indicative of higher risk of elevated ICP and indicates insertion of a ventriculostomy catheter or monitoring device. While a positive ceruloplasmin or copper test result, alone, is predictive, a test showing subnormal levels of both of those markers is a more robust test.

In some embodiments, the level of risk of an at-risk patient who has experienced a possible brain injury event is transformed from being theoretically at risk for deleteriously elevated intracranial pressure to being at known risk for developing elevated intracranial pressure. This is determined by analyzing a test sample obtained from the patient for ceruloplasmin levels, copper levels, or both, and determining that the patient's serum ceruloplasmin levels, serum copper levels, or both, are decreased below their normal ranges. The patient is a human or any other mammal, such as a companion animal (e.g., dog or cat), a show or competition animal (e.g., racing dog or horse) or a farm animal (e.g., horse, cow, sheep, pig), for example.

In some embodiments a process for identifying a patient that is at risk for developing elevated intracranial pressure when the patient has experienced a possible brain injury is provided, which comprises analyzing one or more test sample of body fluid (such as, serum, plasma, blood, urine, cerebral spinal fluid, urine and saliva) obtained from the patient for ceruloplasmin levels, copper level, or both, and determining that the patient's serum ceruloplasmin levels, serum copper levels, or both, fall below the established normal range for the assay method used. A positive test result is an assay result in which the level of the biomarkers ceruloplasmin, copper, or both, in the patient's serum are below the respective established normal ranges for the assay methods used. A positive test result indicates that the patient is at increased risk of elevated ICP.

At a minimum, a positive test with one or more markers would indicate a need for increased frequency and/or intensity of monitoring of the patient. For example, level of consciousness, pupillary response (e.g., size, shape, reaction to light, differences between left and right), visual changes (e.g., in conscious patients, visual acuity, blurred vision, diplopia, field cuts—extraocular motor assessment), motor function, headache (if the patient is conscious), vomiting, changes in blood pressure, pulse, respiratory pattern and temperature may be monitored. Additional symptoms and/or parameters may be monitored, as described elsewhere herein.

One or more additional measures that may be taken to treat a patient who tests positive, as indicated above, including: (a) elevating the head of the patient's bed to about 30 degrees, positioning to maintain neck in neutral position and avoid valsalva maneuver; (b) avoiding hypotension and hypoxemia; (c) maintaining cerebral perfusion pressure (CPP=MAP−ICP) at a minimum of 60 mm Hg. Monitoring of ICP/CPP requires placement of a monitor; (d) draining CSF prn. Draining CSF requires placement of a ventriculostomy; (e) administration of mannitol. Notably, mannitol has been shown to not work prophylactically; (f) administration of hypertonic saline, (g) analgesia and sedation, and (h) neuromuscular blockers. Still other measures may be taken in some cases, including (i) hypothermia, which is currently being studied for its effect on outcome, (j) hyperventilation, which is currently a controversial treatment and generally not recommended unless in acute and emergent situations, and is used for a very short term due to rebound effect, (k) barbiturate coma, which is usually one of the last treatments to be employed, and (l) (l) transport of the patient to a specialized neurotrauma facility, (m) imaging, and (n) neurosurgery to remove hematoma, tumor, or abscess, and/or craniectomy or craniotomy to allow expansion of the brain. Prior to implementation of any of the above-mentioned interventions based on a biomarker screen indicative of ceruloplasmin and/or copper levels, the potential risk of such intervention would also be considered.

Ceruloplasmin is the major copper carrier protein in the blood and plays an important role in copper and iron metabolism (Hellman, N. E. and Gitlin, J. D., 2002. Ceruloplasmin metabolism and function. Annu. Rev. Nutr. 22, 439-458). Serum levels of ceruloplasmin, which is an acute phase protein, have been reported to be increased in response to injury and infection. For example, Rocca, et al. 1987 (Lack of prognostic value of the determination of 3 serum proteins during the acute phase of brain injury. Ann Fr Anesth Reanim. 6(6):476-81, 1987 performed a study designed to determine albuminemia, prealbuminemia, transferrin and ceruloplasmin levels in intensive care unit patients admitted after multiple trauma including head injury. Those investigators determined that in the patients that survived, there was a significant decrease in albuminemia and transferrin levels, no change in prealbuminemia and a significant increase in ceruloplasmin levels. Increases in serum ceruloplasmin and C-reactive protein levels were also reported in brain-injured patients on admission to the hospital by Young, et al. 1988 (The acute-phase response of the brain-injured patient. J Neurosurg. September; 69(3):375-80, 1988). Similarly, Kuhlow, et al. 2003 (Astrocytic ceruloplasmin expression, which is induced by IL-1beta and by traumatic brain injury, increases in the absence of the IL-1 type 1 receptor. Glia. October; 44(1):76-84, 2003) also reported that, in mice, ceruloplasmin levels increased significantly above controls after injury. Further, while not wishing to be limited to any particular theory, it has been postulated that as ceruloplasmin is an acute phase protein, the serum levels of ceruloplasmin increase in response to injury and infection where it is thought to play a protective role due to its antioxidant properties (Goldstein, I. M., Kaplan, H. B., Edelson, H. S. and Weissmann, G., 1982. Ceruloplasmin: an acute phase reactant that scavenges oxygen-derived free radicals. Ann. N.Y. Acad. Sci. 389, 368-379; Tomas, E. and Toparceanu, F. Considerations about the possible function of ceruloplasmin in influenza and parainfluenza virus infections. Virologie. 37, 279-287, 1986; Young, A. B., Ott, L. G., Beard, D., Dempsey, R. J., Tibbs, P. A. and McClain, C. J. The acute-phase response of the brain-injured patient. J. Neurosurg. 69, 375-380, 1988; Rebhun, J., Madorsky, J. G. and Glovsky, M. M. Proteins of the complement system and acute phase reactants in sera of patients with spinal cord injury. Ann. Allergy 66, 335-338, 1991).

Consistent with the postulated protective role for ceruloplasmin are diseases associated with decreased ceruloplasmin levels, for example Wilson's Disease, Menke's Syndrome, and ceruloplasminea which cause dementia, liver failure, and other pathological features (Menkes, J. H., 1988. Kinky hair disease: twenty five years later. Brain Dev. 10, 77-79; Xu, X., Pin, S., Gathinji, M., Fuchs, R. and Harris, Z. L., 2004. Aceruloplasminemia: an inherited neurodegenerative disease with impairment of iron homeostasis. Ann. N.Y. Acad. Sci. 1012, 299-305; Vassiliev, V., Harris, Z. L. and Zatta, P., 2005. Ceruloplasmin in neurodegenerative diseases. Brain Res. Brain Res. Rev. 49, 633-640; Das, S. K. and Ray, K., 2006. Wilson's disease: an update. Nat. Clin. Pract. Neurol. 2, 482-493).

Copper (Cu++) is an essential mineral that is obtained through diet. It is absorbed in the intestines and transported to the liver, where it is stored and used in the production of a variety of enzymes and metaloproteins. The liver binds copper to apoceruloplasmin to produce ceruloplasmin and then releases it into the bloodstream. Normally it is believed that most of the copper in the blood is bound to ceruloplasmin. Because of this, ceruloplasmin assays are used along with one or more copper tests to aid in the evaluation of a patients' copper metabolism and ceruloplasmin levels. These values are used to aid in the diagnosis and prognosis of Wilson's disease, Menke's syndrome and ceruloplasminemia.

Wilson's disease is a rare autosomal recessive inherited disorder. Wilson's disease causes the body to take in and retain too much copper and is characterized by decreased ceruloplasmin. The copper deposits in the liver, brain, kidneys, and the eyes where patients develop rusty or brown-colored rings around the iris known as Kayser-Fleischer rings. These copper deposits cause tissue damage, scarring, death of the tissues and eventually lead to organ malfunction. Liver failure and damage to the central nervous system (brain, spinal cord) are the most common, and the most dangerous, effects of the disorder. If not detected and treated early, Wilson's disease is fatal.

Menke's syndrome (also known as steely hair disease; Menke's kinky hair syndrome) is an inherited x-linked recessive condition that results from a defect in the ATP7A gene which makes it difficult for the body to distribute and absorb copper. As a result, the brain and other parts of the body do not obtain the copper required. Menke's syndrome is very severe and most individuals with this condition die within the first few years of life.

These rare genetic disorders along with a dietary copper deficiency can result in decreased blood and urine copper concentrations as well as decreased ceruloplasmin levels. Thus, these conditions represent possible confounders for the interpretation of the results of a test because they will result in a lowering of the patient's baseline ceruloplasmin and/or copper level, thus this may potentially misdirect the practitioner by leading them to believe that the trauma patient appears to be at risk for elevated ICP when they are not (a false positive). Thus, in some cases, copper deficiency and these, albeit, rare metabolic disorders are considered, when appropriate, during differential diagnosis to identify those who will develop elevated ICP, particularly in young patients. It should, however, be noted that both Wilson's disease and Menke's syndrome are severe, so it is very likely that patients with these diseases and their families will be aware of their pre-existing condition. Furthermore, as Menke's syndrome is fatal within the first few years of life, its consideration is likely to be limited to the use of a disclosed method to identify a patient who will develop elevated ICP during brain injury in infants and young children, for example, but not limited to, those with TBI due to accidental trauma or those suffering from shaken baby syndrome.

### Ceruloplasmin Proteins and Amino Acid Sequences

Ceruloplasmin proteins, polypeptides, peptides and fragments thereof can be used to identify the levels of ceruloplasmin in body fluids (such as but not limited to, serum, plasma, blood, CSF, urine, and saliva) as a way to determine, estimate or predict serum ceruloplasmin levels, using mass spectrometry, similar to that described in Example 1.3, below. Many complete, partial, predicted and other amino acid sequences for Human (Homo sapiens) include but are not limited to, those described in, for example, various database accession numbers: NP_{—}000087, BAA08084, BAA08085, AAF02483 AAA40914, AAA40915, AAA51975, P00450, BAA00019 (proceruloplasmin), EAW78882.1 and AA51976 (preceruloplasmin, EC 1.16.3.1). Human ceruloplasmin is also described by Yang, et al., 1986 (“Characterization, mapping, and expression of the human ceruloplasmin gene.” Proc. Natl. Acad. Sci. U.S.A. 83 (10), 3257-3261, 1986). Veterinary care of companion and some large animals represents a large and growing market. Accordingly, in some veterinary applications, animals at risk for elevated ICP are identified by determining a reduced level of serum ceruloplasmin in the animal. For example, ceruloplasmin proteins, polypeptides, peptides and fragment levels may also be used to identify the levels of ceruloplasmin in body fluids including, but not limited to serum, plasma, blood, cerebral spinal fluid (CSF), urine, and saliva obtained from companion animals, racing animals or other mammals. In some embodiments, ceruloplasmin levels in fluids other than serum are used as surrogate markers for determining, estimating or predicting serum levels in the mammal. Because ceruloplasmin sequences, actual and predicted have also been obtained from experimental animals, companion animals, farm animals and even various aquatic animals, the use of mass spectrometry similar to that described in Example 1.3, is also possible with any suitable animals. These ceruloplasmin sequences include, but are not limited to database accession numbers: XP_{—}001103724, XP_{—}001109462, XP_{—}001109219, and XP_{—}001109373 (rhesus monkey (Macaca mulatta); NP_{—}036664, AAB65820, AAF34175, P13635 (rat, Rattus norvegicus); AAB07996, AAH62957, Q61147, NP_{—}001036076, NP_{—}031778, ABC68391 (mouse, Mus musculus); AAS77867, XP_{—}534301, XP_{—}854412, XP_{—}865885, XP_{—}865901 (dog, Canis familiaris); AAF31300 (horse, Equus caballus) XP_{—}604593, XP_{—}592003 (cow, Bos taurus); NP_{—}001009733, AAD41477, Q9XT27 (sheep, Ovis aries); ACD93403 (wild boar, Sus scrofa); ACO82077 (Ibex, capra ibex); XP_{—}001508579, XP_{—}00150540 (platypus, Ornithorhynchus anatinus); XP_{—}001371162, XP_{—}001371118 (opossum, Monodelphis domestica); XP_{—}001235149 (chicken, Gallus gallus); NP_{—}571877, AAK55535, AAH48037, AAH64000, CAK04907, CAK04908, CAK04909 (zebra fish: Danio rerio); CAL92184 (ocellated icefish, Chionodraco rastrospinosus); ACO51200 (bighead carp, Aristichthys nobilis); AAT01920 (Winter flounder, Pseudopleuroectens americanus); XP_{—}001192054, XP_{—}001198170 (sea urchin, Strongylocentrotus purpuratus).

In various embodiments, ceruloplasmin expression products, proteins, polypeptides, peptide fragments, mutated, truncated, or deleted forms of ceruloplasmin, and/or ceruloplasmin fusion proteins, are prepared for a variety of uses including, but not limited to, the generation of antibodies, as reagents in diagnostic assays which are then used to identify individuals at risk of developing elevated ICP.

Ceruloplasmin amino acid sequences that may be used in various applications include the ceruloplasmin amino acid sequences described herein, as well as analogues and derivatives thereof. Further, use of corresponding ceruloplasmin orthologs and homologs from other species are also envisioned for some applications.

Such functionally equivalent ceruloplasmin amino acid sequences include, but are not limited to, additions or substitutions of amino acid residues within the amino acid sequences of ceruloplasmin described herein, but those that result in a silent change, thus producing a functionally equivalent gene product. Amino acid substitutions may be made on the basis of similarity in polarity, charge, solubility, hydrophobicity, hydrophilicity, and/or the amphipathic nature of the residues involved. For example: nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Amino acid substitutions may alternatively be made on the basis of the hydropathic index of amino acids. Each amino acid can be assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The use of the hydropathic amino acid index in conferring interactive biological function on a protein is understood in the art (Kyte and Doolittle, J. Mol. Biol. 157:105-132, 1982). It is known that in certain instances, certain amino acids may be substituted for other amino acids having a similar hydropathic index or score and still retain a similar biological activity. In making changes based upon the hydropathic index, in certain embodiments the substitution of amino acids whose hydropathic indices are within 2 is included, while in other embodiments amino acid substitutions that are within 1 are included, and in yet other embodiments amino acid substitutions within 0.5 are included.

Amino acid substitutions may alternatively be made on the basis of hydrophilicity, particularly where the biologically functional protein or peptide thereby created is intended for use in immunological embodiments. In certain embodiments, the greatest local average hydrophilicity of a protein, as governed by the hydrophilicity of its adjacent amino acids, correlates with its immunogenicity and antigenicity, i.e., with a biological property of the protein. The following hydrophilicity values have been assigned to these amino acid residues: arginine (+3.0); lysine (+3.0); aspartate (+3.0 1); glutamate (+3.0 1); serine (+0.3); asparagine (+0.2); glutamine (+0.2); glycine (0); threonine (−0.4); proline (−0.5 1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments the substitution of amino acids whose hydrophilicity values are within 2 is included, in certain embodiments those that are within 1 are included, and in certain embodiments those within 0.5 are included. One may also identify epitopes from primary amino acid sequences on the basis of hydrophilicity.

For example, the amino acid sequence of human ceruloplasmin peptides, polypeptides, and proteins can be aligned with homologs from different species. Mutant peptides, polypeptides, and proteins can be engineered so that regions of interspecies identity are maintained, whereas the variable residues are altered, e.g., by deletion or insertion of an amino acid residue(s) or by substitution of one or more different amino acid residues. For example, alterations in variable residues may be designed to produce a mutant form of a ceruloplasmin peptide, polypeptide, or protein that is more stable but retains function.

### Ceruloplasmin Peptide Epitopes and Immunogens

The term “epitope” refers to any polypeptide determinant capable of selectively binding to an immunoglobulin or a T-cell receptor. In general, an epitope is a region of an antigen that is selectively bound by an antibody. In certain cases, an epitope may include chemically active surface groupings of molecules such as amino acids, sugar side chains, phosphoryl, and/or sulfonyl groups. Additionally, an epitope may have specific three dimensional structural characteristics (e.g., a “conformational” epitope) and/or specific charge characteristics.

An epitope is defined as “the same” as another epitope if a particular antibody selectively binds to both epitopes. In certain cases, polypeptides having different primary amino acid sequences may comprise epitopes that are the same, and epitopes that are the same may have different primary amino acid sequences. Different antibodies are said to bind to the same epitope if they compete for selective binding to that epitope.

One may identify epitopes from primary amino acid sequences on the basis of hydrophilicity, as described above. These regions are also referred to as “epitopic core regions.” In general, ceruloplasmin peptides selected for immunizing an animal comprise one or more epitopes, as such peptides are likely to be immunogenic. In general, peptide immunogens and epitopes are those that are predicted to be hydrophilic and/or likely to be exposed on the surface of native ceruloplasmin in its folded state. In certain embodiments, peptide segments that are predicted to form β-turns, and are therefore likely to be exposed on the surface of a protein, may be selected as immunogens. Alternatively, it is not necessary that the epitope be expressed on the surface of the protein. Many immunological techniques utilize the addition of reagents to facilitate protein unfolding, thereby unmasking epitopes that were unavailable prior to the manipulation. Guidance for selecting suitable immunogenic peptides and related techniques are provided, for example, in “Current Protocols in Molecular Biology”, Vol. 1 and 2 (Ausubel et al., eds., Green Publishing Associates, Incorporated, and John Wiley & Sons, Incorporated, New York, N.Y., 1989) Ch. 11.14, and “Antibodies: A Laboratory Manual” (Harlow and Lane, eds., Cold Spring Harbor Laboratory Press, Cold Spring Harbor, N.Y., 1988) Ch. 5.

Certain algorithms are known to those skilled in the art for predicting whether a peptide segment of a protein is hydrophilic, and therefore likely to be exposed on the surface of the protein. These algorithms use the primary sequence information of a protein to make such predictions, and are based on the method of, for example, Hopp and Woods, Proc. Natl. Acad. Sci. USA 78:3824-3828, 1981, or Kyte and Doolittle, supra. Certain exemplary algorithms are known to those skilled in the art for predicting the secondary structure of a protein based on the primary amino acid sequence of the protein (see, e.g., Corrigan and Huang, Comput. Programs Biomed. 15:163-168, 1982, Chou and Fasman, Ann. Rev. Biochem. 47:251-276, 1978, Moult, Curr. Opin. Biotechnol. 7:422-427, 1996, Chou and Fasman, Biochemistry 13:222-245, 1974, Chou and Fasman, Biochemistry 13:211-222, 1974, Chou and Fasman, Adv. Enzymol. Relat. Areas Mol. Biol. 47:45-148, 1978, and Chou and Fasman, Biophys. J. 26:367-383, 1979).

Moreover, computer programs are currently available to assist with predicting secondary structure. One method of predicting secondary structure is based upon homology modeling. For example, two polypeptides or proteins that have a sequence identity of greater than 30%, or similarity greater than 40%, often have similar structural topologies. The growth of the Protein Structural Database (PSDB); Berman et al., Nucleic Acids Res. 28:235-242, 2000) and the Protein Data Bank (PDB) has provided enhanced predictability of secondary structure, including the potential number of folds within the structure of a polypeptide (see, e.g., Holm and Sander, Nucleic Acids Res. 27:244-247, 1999). It has been suggested there are a limited number of folds in a given polypeptide or protein, and once a critical number of structures have been resolved, structural prediction will become much more accurate (Brenner et al., Curr. Opin. Struct. Biol. 7:369-376, 1997). Additional methods of predicting secondary structure include “threading” (see, e.g., Jones, Curr. Opin. Struct. Biol. 7:377-387, 1997, and Sippl and Flockner, Structure 4:15-19, 1996), “profile analysis” (see, e.g., Bowie et al., Science 253:164-170, 1991, Gribskov et al., Meth. Enzymol. 183:146-159, 1990, and Gribskov et al., Proc. Natl. Acad. Sci. USA 84:4355-4358, 1987), and “evolutionary linkage” (see, e.g., Holm and Sander, 1999, supra, and Brenner et al., 1997, supra).

### Antibodies to Ceruloplasmin Proteins

The use of antibodies that selectively bind to one or more epitopes of ceruloplasmin or epitopes of conserved variants of ceruloplasmin and its fragments are also contemplated, particularly for use in the immunoassays described herein. Antibodies for use in these immunoassays include those available commercially from, Abcam Inc., (Cambridge, Mass.), AbD Serotec (a division of MorphoSys US Inc. Raleigh, N.C.), ABR-Affinity Bioreagents (now Thermo Fisher Scientific-Rockford, Ill.), Santa Cruz Biotechnology (Santa Cruz, Calif.), DAKO (Carpinteria, Calif.), GenWay Biotech (San Diego, Calif.) and BD Biosciences (San Jose, Calif.), for example. In addition anti-human ceruloplasmin monoclonal antibodies are described, at least, in U.S. Pat. Nos. 5,491,066 and 6,010,903.

Such antibodies include, but are not limited to, polyclonal antibodies, monoclonal antibodies (mAbs), humanized antibodies, human-engineered antibodies, fully human antibodies, chimeric antibodies, single chain antibodies, Fab fragments, F(ab′)₂ fragments, fragments produced by a Fab expression library, anti-idiotypic (anti-Id) antibodies, catalytic antibodies, and epitope-binding fragments of any of the above. In some applications, the antibodies, or fragments thereof, will preferentially bind to ceruloplasmin, as opposed to other oxidase proteins. In such cases, the antibodies, or fragments thereof, selectively bind to ceruloplasmin with a higher affinity or avidity than they bind to other oxidase proteins.

An antibody “selectively binds” an antigen when it preferentially recognizes the antigen in a complex mixture of proteins and/or other macromolecules. The antibodies employed in some of the methods disclosed herein comprise an antigen-binding site that selectively binds to a particular epitope. Such antibodies can be capable of binding to different antigens, so long as the different antigens comprise that particular epitope. In some applications, homologous proteins from different species comprise the same epitope. In various applications, an antibody selectively binds an antigen when the dissociation constant (K_{D}) is 1 uM, or when the dissociation constant is 100 nM, or when the dissociation constant is 10 nM, for example.

Antibodies that selectively bind to ceruloplasmin may be used, for example, in the detection and determination of ceruloplasmin levels in a biological sample and may, therefore, be utilized as part of a diagnostic or prognostic technique whereby patients may be tested to determine if normal or abnormal amounts of ceruloplasmin are present. In some embodiments, these antibodies are used in the ceruloplasmin immunoassays described herein, and in the identification and quantitation of the level of ceruloplasmin in the body fluids of patients with disorders associated with elevated ICP in order to identify those at increased risk of developing elevated ICP.

A native antibody typically has a tetrameric structure comprising two identical pairs of polypeptide chains, each pair having one light chain (typically about 25 kDa) and one heavy chain (typically about 50-70 kDa). In a native antibody, a heavy chain comprises a variable region, V_{H}, and three constant regions, C_{H}1, C_{H}2, and C_{H}3. The V_{H} domain is at the amino-terminus of the heavy chain, and the C_{H}3 domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In humans, for example, native human light chains are typically classified as kappa and lambda light chains. Native human heavy chains are typically classified as mu, delta, gamma, alpha, or epsilon, and define the isotype of the antibody as IgM, IgD, IgG, IgA, and IgE, respectively. IgG has subclasses, including, but not limited to, IgG1, IgG2, IgG3, and IgG4. IgM has subclasses including, but not limited to, IgM1 and IgM2. IgA has subclasses including, but not limited to, IgA1 and IgA2. Within native human light and heavy chains, the variable and constant regions are typically joined by a “J” region of about 12 or more amino acids, with the heavy chain also including a “D” region of about 10 more amino acids (“Fundamental Immunology”, 2^{nd} Ed., Ch. 7 (Paul, ed., Raven Press, New York, N.Y., 1989)). In various applications, the antibodies used in an immunoassay are of any of the isotypes or isotype subclasses set forth above.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen binding site. For example, H3, in certain instances, can be as short as two amino acid residues or greater than 26. The assignment of amino acids to each domain is typically in accordance with the definitions in “Sequences of Proteins of Immunological Interest” (Kabat et al., eds., National Institutes of Health, Publication No. 91-3242, 5^{th} Ed., United States Department of Health and Human Services, Bethesda, Md., 1991), Chothia and Lesk, J. Mol. Biol. 196:901-917, 1987, or Chothia et al., Nature 342:878-883, 1989. In the present application, the term “CDR” refers to a CDR from either the light or heavy chain, unless otherwise specified.

### Production of Ceruloplasmin Antibodies

In addition to ceruloplasmin antibodies and ceruloplasmin assay kits, as are known to those of skill in the art and may be commercially available, antibodies for use in the ceruloplasmin immunoassays disclosed herein include those that are generated de novo.

For the production of antibodies, various host animals, such as but not limited to chickens, hamsters, guinea pigs, rabbits, sheep, goats, horses, may be immunized by injection with a ceruloplasmin protein, polypeptide, or peptide, a truncated ceruloplasmin polypeptide, a functional equivalent of ceruloplasmin, a mutant of ceruloplasmin, an antigenic fragment thereof, or combinations thereof. Such host animals may include, but are not limited to, rabbits, mice, and rats, and ceruloplasmin “knock-out” variants of the same. In addition, antibodies can be produced by immunizing female birds (chickens, for example) and harvesting the IgY antibodies present in their eggs. Various adjuvants may be used to increase the immunological response, depending on the host species, including, but not limited to, Freund's adjuvant (complete and incomplete), mineral salts such as aluminum hydroxide or aluminum phosphate, surface active substances, chitosan, lysolecithin, pluronic polyols, polyanions, peptides, oil emulsions, and potentially useful human adjuvants such as BCG (Bacille Calmette-Guerin) and Corynebacterium parvum. Alternatively, the immune response could be enhanced by combination and/or coupling with molecules such as keyhole limpet hemocyanin (KLH), tetanus toxoid, diphtheria toxoid, ovalbumin, cholera toxin, or fragments thereof. Alternatively expression as a fusion protein, such as GST, His6, or another suitable fusion protein may be used.

Polyclonal antibodies are heterogeneous populations of antibody molecules, such as those derived from the sera of the immunized animals or by mixing B-cells or monoclonal antibodies. Monoclonal antibodies, which are homogeneous populations of antibodies that arise from a single B-cell or its which selectively bind to a particular antigen or epitope, may be obtained by any technique that provides for the production of antibody molecules by continuous cell lines in culture. These include, but are not limited to, the hybridoma technique (Kohler and Milstein, Nature 256:495-497, 1975, U.S. Pat. No. 4,376,110, and “Antibodies: A Laboratory Manual”, supra, Ch. 6), the human B-cell hybridoma technique (Kozbor and Roder, Immunol. Today 4:72-79, 1983, and Cote et al., Proc. Natl. Acad. Sci. USA 80:2026-2030, 1983), and the EBV-hybridoma technique (Cole et al., Mol. Cell. Biochem. 62:109-120, 1984, and Cole et al., Cancer Res. 44:2750-2753, 1984). A suitable animal, such as a mouse, rat, hamster, monkey, or other mammal, or an avian species, is immunized with an immunogen to produce antibody-secreting cells, including, but not limited to, B-cells, such as lymphocytes or splenocytes. In certain embodiments, lymphocytes (e.g., human lymphocytes) are immunized in vitro to generate antibody-secreting cells (Borrebaeck et al., Proc. Natl. Acad. Sci. USA 85:3995-3999, 1988). The hybridomas producing the monoclonal antibodies that are used in certain embodiments may be cultivated in vitro or in vivo. In some instances, the production of high titer monoclonal antibodies in vivo is the preferred method of producing antibodies for use in a testing method described herein.

For some applications, antibody-secreting cells are fused with an “immortalized” cell line, such as a myeloid-type cell line, to produce hybridoma cells. Hybridoma cells that produce the desired antibodies can be identified, for example, by ELISA, and can then be subcloned and cultured using standard methods, or grown in vivo as ascites tumors in a suitable animal host. For some applications, monoclonal antibodies are isolated from hybridoma culture medium, serum, or ascites fluid using standard separation procedures, such as affinity chromatography (see, e.g., “Antibodies: A Laboratory Manual”, supra, Ch. 8).

For assaying ceruloplasmin levels, in some cases high affinity antibodies are generated using animals that have been genetically engineered to be deficient in ceruloplasmin production and activity. An example of such knock-out animals (mice) are produced using established gene trapping methods, and viable animals that are genetically homozygous for the genetically engineered ceruloplasmin mutation are generated and characterized. Given the relatedness of mammalian ceruloplasmin amino acid sequences, the presently described homozygous knock-out mice (having never seen, and thus never been tolerized to, ceruloplasmin) can be advantageously applied to the generation of antibodies against mammalian ceruloplasmin sequences (i.e., ceruloplasmin will be immunogenic in ceruloplasmin homozygous knock-out animals). High affinity anti-ceruloplasmin antibodies generated from such animals can be formulated into immunoassays that are used, as described herein, to identify those patients at risk for deleteriously elevated ICP.

For example, human monoclonal antibodies are raised in transgenic animals (e.g., mice) that are capable of producing human antibodies (see, e.g., U.S. Pat. Nos. 6,075,181 and 6,114,598, and PCT Patent Application Publication No. WO 98/24893). Human immunoglobulin genes can be introduced (e.g., using yeast artificial chromosomes, human chromosome fragments, or germline integration) into mice in which the endogenous Ig genes have been inactivated (see, e.g., Jakobovits et al., Nature 362:255-258, 1993, Tomizuka et al., Proc. Natl. Acad. Sci. USA 97:722-727, 2000, and Mendez et al., Nat. Genet. 15:146-156, 1997, describing the XenoMouse II® line of transgenic mice), for instance. Additional exemplary methods and transgenic mice suitable for the production of human monoclonal antibodies are described, e.g., in Jakobovits, Curr. Opin. Biotechnol. 6:561-566, 1995, Lonberg and Huszar, Int. Rev. Immunol. 13:65-93, 1995, Fishwild et al., Nat. Biotechnol. 14:845-851, 1996, Green, J. Immunol. Methods 231:11-23, 1999, and Little et al., Immunol. Today 21:364-370, 2000.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985), for example by splicing the genes from a mouse antibody molecule of appropriate antigen selectivity together with genes from a human antibody molecule of appropriate biological activity, can be used. A chimeric antibody is a molecule in which different portions are derived from different animal species, such as those having a variable region derived from a murine monoclonal antibody and a human immunoglobulin constant region. Such technologies are described in U.S. Pat. Nos. 6,075,181 and 5,877,397, for example.

Monoclonal antibodies that are employed in some applications for determining ceruloplasmin levels can also be produced by recombinant techniques (see, e.g., U.S. Pat. No. 4,816,567). In such embodiments, nucleic acids encoding monoclonal antibody chains are cloned and expressed in a suitable host cell. For example, RNA can be prepared from cells expressing the desired antibody, such as mature B-cells or hybridoma cells, which can then be used to make cDNA, using standard methods. The cDNA encoding a heavy or light chain polypeptide can be amplified, for example, by PCR, using specific oligonucleotide primers. The cDNA can then be cloned into a suitable expression vector, which is then transformed or transfected into a suitable host cell, such as a host cell that does not endogenously produce antibody.

Transformation or transfection can be accomplished by any known method suitable for introducing polynucleotides into a host cell. Certain exemplary methods include, but are not limited to, packaging the polynucleotide in a virus (or into a viral vector) and transducing a host cell with the virus (or vector) and using certain transfection procedures known in the art, as exemplified by U.S. Pat. Nos. 4,399,216, 4,912,040, 4,740,461, and 4,959,455. In certain embodiments, the transformation procedure used may depend upon the host to be transformed. Various methods for introduction of heterologous polynucleotides into mammalian cells are known in the art and include, but are not limited to, dextran-mediated transfection, calcium phosphate precipitation, polybrene-mediated transfection, protoplast fusion, electroporation, encapsulation of the polynucleotide(s) in liposomes, and direct microinjection of the DNA into nuclei. In embodiments where heavy and light chains are co-expressed in the same host, reconstituted antibody may be isolated.

Alternatively, techniques described for the production of single chain antibodies (Bird et al., Science 242:423-426, 1988, Huston et al., Proc. Natl. Acad. Sci. USA 85:5879-5883, 1988, Ward et al., Nature 341:544-546, 1989, PCT Patent Application Publication No. WO 88/01649, and U.S. Pat. Nos. 4,946,778 and 5,260,203) can be adapted to produce single chain antibodies against ceruloplasmin gene products or epitopes. Single chain antibodies are formed by linking the heavy and light chain fragments of the Fv region via an amino acid bridge, resulting in a single chain polypeptide with an antigen binding region.

In some applications, a method or test kit disclosed herein for measuring ceruloplasmin levels employs antibody fragments, including, but not limited to, Fab, Fab′, F(ab′)₂, Fv, scFv, Fd, diabodies, and other antibody fragments that retain at least a portion of the variable region of an intact antibody (see, e.g., Hudson and Souriau, Nature Med. 9:129-134, 2003). A Fab fragment comprises one light chain and the C_{H}1 and variable region of one heavy chain. The heavy chain of a Fab molecule cannot form a disulfide bond with another heavy chain molecule. A Fab′ fragment comprises one light chain and one heavy chain that comprises additional constant region, extending between the C_{H}1 and C_{H}2 domains, and can be generated by reducing the disulfide bridges of F(ab′)₂ fragments. An interchain disulfide bond can be formed between two heavy chains of a Fab′ fragment to form a F(ab′)₂ molecule, which can be produced by pepsin digestion of an antibody molecule. A Fv fragment comprises the variable regions from both the heavy and light chains, but lacks the constant regions. In certain instances, a single variable region (one-half of a Fv) may have the ability to recognize and bind antigen, albeit with lower affinity than the Fv. A Fab expression library may also be constructed (Huse et al., Science 246:1275-1281, 1989) to allow rapid and easy identification of monoclonal Fab fragments with the desired selectivity.

Monoclonal antibodies employed in certain embodiments may also be produced using a display-based method. For example, monoclonal antibodies can be produced using phage display techniques (see, e.g., Hoogenboom, Methods Mol. Biol. 178:1-37, 2002, Clackson et al., Nature 352:624-628, 1991, and Marks et al., J. Mol. Biol. 222:581-597, 1991). For example, a library of antibodies can be displayed on the surface of a filamentous phage, such as the nonlytic filamentous phage fd or M13. The antibodies can be antibody fragments, such as scFvs, Fabs, Fvs with an engineered intermolecular disulfide bond to stabilize the V_{H}-V_{L} pair, and diabodies. Using these techniques, antibodies with the desired binding selectivity can then be selected.

For example, in some instances, variable gene repertoires are prepared by PCR amplification of genomic DNA or cDNA derived from the mRNA of antibody-secreting cells, such as B-cells. For example, cDNA encoding the variable regions of heavy and light chains can be amplified by PCR, and the heavy chain cDNA and light chain cDNA cloned into a suitable vector. The heavy chain cDNA and light chain cDNA can be randomly combined during the cloning process, thereby resulting in the assembly of a cDNA library encoding diverse scFvs or Fabs. Alternatively, the heavy chain cDNA and light chain cDNA can be ligated, for example by stepwise cloning, before being cloned into a suitable vector.

Suitable vectors include, but are not limited to, phage display vectors, such as a phagemid vectors. Certain exemplary phagemid vectors, such as pCES1, are known to those skilled in the art. In certain embodiments, cDNA encoding both heavy and light chains is present on the same vector. For example, cDNA encoding scFvs can be cloned in-frame with all or a portion of gene III, which encodes the minor phage coat protein pIII. The phagemid then directs the expression of the scFv-pIII fusion on the phage surface. Alternatively, cDNA encoding heavy chain (or light chain) can be cloned in-frame with all or a portion of gene III, and cDNA encoding light chain (or heavy chain) can be cloned downstream of a signal sequence in the same vector. The signal sequence directs expression of the light chain (or heavy chain) into the periplasm of the host cell, where the heavy and light chains assemble into Fab fragments. In other methods, cDNA encoding heavy chain and cDNA encoding light chain can be present on separate vectors. In these methods, heavy chain and light chain cDNA are cloned separately, one into a phagemid and the other into a phage vector, which both contain signals for in vivo recombination in the host cell. The recombinant phagemid and/or phage vectors are introduced into a suitable bacterial host, such as E. coli. When using certain phagemids, the host can be infected with helper phage to supply phage structural proteins, thereby allowing expression of phage particles carrying the antibody-pIII fusion protein on the phage surface.

“Synthetic” antibody libraries can be constructed using repertoires of variable genes that are rearranged in vitro. For example, individual gene segments encoding heavy or light chains (V-D-J or V-J, respectively) are randomly combined using PCR. Additional sequence diversity can be introduced into the CDRs, such as CDR3 (H3 of the heavy chain), and possibly FRs, by error prone PCR.

“Naïve” or “universal” phage display libraries can be constructed, as described above, using nucleic acids from a naïve (unimmunized) animal, while “immunized” phage display libraries can be constructed, as described above, using nucleic acids from an immunized animal. Exemplary universal human antibody phage display libraries are available from commercial sources, and include, but are not limited to, the HuCAL® series of libraries from MorphoSys AG (Martinstried/Planegg, Germany), libraries from Crucell (Leiden, the Netherlands) using MAbstract® technology, the n-CoDeR™ Fab library from BioInvent International AB (Lund, Sweden), and libraries available from Cambridge Antibody Technology (Cambridge, United Kingdom).

Selection of antibodies having the desired binding selectivity from a phage display library can be achieved by successive panning steps. In panning, library phage preparations are exposed to one or more antigen(s), such as one or more ceruloplasmin antigen(s). The phage-antigen complexes are then washed, and unbound phage are discarded. The bound phage are recovered, and subsequently amplified by infecting E. coli. Monoclonal antibody-producing phage can be cloned by picking single plaques. In some instances, the above process is repeated one or more times.

The antigen is immobilized on a solid support to allow purification of antigen-binding phage by affinity chromatography. Alternatively, the antigen is biotinylated, thereby allowing the separation of bound phage from unbound phage using streptavidin-coated magnetic beads. In some instances, the antigen is immobilized on cells (for direct panning), in tissue cryosections, or on membranes (e.g., nylon or nitrocellulose membranes). Other variations of these panning procedures may be routinely determined by one skilled in the art.

Yeast display systems may also be used to produce monoclonal antibodies. In these systems, an antibody is expressed as a fusion protein with all or a portion of a yeast protein, for example the yeast AGA2 protein, which becomes displayed on the surface of the yeast cell wall. Yeast cells expressing antibodies with the desired binding selectivity can then be identified by exposing the cells to fluorescently labeled antigen, and isolated by flow cytometry (see, e.g., Boder and Wittrup, Nat. Biotechnol. 15:553-557, 1997).

### Modified Ceruloplasmin Antibodies

Antibodies for use in immunoassays used to determine ceruloplasmin levels in the body fluids of those at risk for elevated ICP may include antibodies that are modified to alter one or more of the properties of the antibody. For some applications, a modified antibody may possess certain advantages over an unmodified antibody, such as increased affinity, for example. An antibody can be modified by linking it to a nonproteinaceous moiety, or by altering the glycosylation state of the antibody, e.g., by altering the number, type, linkage, and/or position of carbohydrate chains on the antibody, or altered so that it is not glycosylated.

In some other modification techniques, one or more chemical moieties may be linked to the amino acid backbone and/or carbohydrate residues of the antibody. Certain exemplary methods for linking a chemical moiety to an antibody include, but are not limited to, acylation reactions or alkylation reactions (see, e.g., Malik et al., Exp. Hematol. 20:1028-1035, 1992, Francis, in “Focus on Growth Factors”, Vol. 3, No. 2, pp. 4-10 (Mediscript, Ltd., London, United Kingdom, 1992), European Patent Application Publication Nos. EP 0 401 384 and EP 0 154 316, and PCT Patent Application Publication Nos. WO 92/16221, WO 95/34326, WO 95/13312, WO 96/11953, and WO 96/19459). These reactions may be used to generate an antibody that is chemically modified at its amino-terminus for use in certain embodiments.

An antibody may also be modified by linkage to a detectable label, such as an enzymatic, fluorescent, isotopic or affinity label. Such a detectable label may allow for the detection or isolation of the antibody, and/or the detection of an antigen bound by the antibody in various immunoassays. Depending on the nature of the label, qualitative and/or quantitative determinations of ceruloplasmin levels can be made using a colorimeter, a spectrophotometer, an ELISA reader, a fluorometer, or a gamma or scintillation (alpha or beta) counter that detects radioactive decay in assays utilizing isotope labels.

### Affinity Maturation of Ceruloplasmin Antibodies

Higher affinity ceruloplasmin antibodies are employed to provide significant advantages in certain embodiments of the ceruloplasmin immunoassays described herein. Potential advantages include, but are not limited to, greater assay sensitivity, increased linearity, and decreased cost of goods. Antibody affinity may, in some cases, determine the formats that are available for use in an ceruloplasmin immunoassays.

The affinity of an antibody for a particular antigen may be increased by subjecting the antibody to affinity maturation (or “directed evolution”) in vitro. In vivo, native antibodies undergo affinity maturation through somatic hypermutation followed by selection. Certain in vitro methods mimic that in vivo process, thereby allowing the production of antibodies having affinities that equal or surpass that of native antibodies.

In certain types of affinity maturation, mutations are introduced into a nucleic acid sequence encoding the variable region of an antibody having the desired binding selectivity (see, e.g., Hudson and Souriau, supra, and Brekke and Sandlie, Nat. Rev. Drug Discov. 2:52-62, 2002). Such mutations can be introduced into the variable region of the heavy chain, light chain, or both, into one or more CDRs, into H3, L3, or both, and/or into one or more FRs. A library of mutations can be created, for example, in a phage, ribosome, or yeast display library, so antibodies with increased affinity may be identified by standard screening methods (see, e.g., Boder et al., Proc. Natl. Acad. Sci. USA 97:10701-10705, 2000, Foote and Eisen, Proc. Natl. Acad. Sci. USA 97:10679-10681, 2000, Hoogenboom, supra, and Hanes et al., Proc. Natl. Acad. Sci. USA 95:14130-14135, 1998).

Mutations can be introduced by site-specific mutagenesis, based on information on the structure of the antibody, e.g., the antigen binding site, or using combinatorial mutagenesis of CDRs. Alternatively, all or a portion of the variable region coding sequence may be randomly mutagenized, e.g., using E. coli mutator cells, homologous gene rearrangement, or error prone PCR. Mutations may also be introduced using “DNA shuffling” (see, e.g., Crameri et al., Nature Med. 2:100-102, 1996, and Fermer et al., Tumour Biol. 25:7-13, 2004).

In addition, “chain shuffling” may be used to generate antibodies with increased affinity. In chain shuffling, one of the chains, e.g., the light chain, is replaced with a repertoire of light chains, while the other chain, e.g., the heavy chain, is unchanged, thus providing selectivity. A library of chain shuffled antibodies can be created, wherein the unchanged heavy chain is expressed in combination with each light chain from the repertoire of light chains. Such libraries may then be screened for antibodies with increased affinity. In particular applications, both the heavy and light chains are sequentially replaced, only the variable regions of the heavy and/or light chains are replaced, or only a portion of the variable regions, e.g., CDRs, of the heavy and/or light chains are replaced (see, e.g., Hudson and Souriau, supra, Brekke and Sandlie, supra, Kang et al., Proc. Natl. Acad. Sci. USA 88:11120-11123, 1991, and Marks et al., Biotechnology (NY) 10:779-783, 1992).

Mouse monoclonal antibodies that selectively bind human ceruloplasmin or ceruloplasmin from other mammals are subject to sequential chain shuffling. Such monoclonal antibodies include but not limited to, mouse monoclonal antibodies raised against mouse ceruloplasmin but selectively bind to (i.e., cross-react with) human ceruloplasmin. For example, the heavy chain of a given mouse monoclonal antibody may be combined with a new repertoire of human light chains, and antibodies with the desired affinity may be selected. The light chains of the selected antibodies may then be combined with a new repertoire of human heavy chains, and antibodies with the desired affinity may be selected. In this manner, human antibodies having the desired antigen binding selectivity and affinity are obtained.

Alternatively, the heavy chain of a given mouse monoclonal antibody may be combined with a new repertoire of human light chains, and antibodies with the desired affinity selected from this first round of shuffling. In addition, the light chain of the original mouse monoclonal antibody is combined with a new repertoire of human heavy chains, and antibodies with the desired affinity selected from this second round of shuffling. Then, human light chains from the antibodies selected in the first round of shuffling are combined with human heavy chains from the antibodies selected in the second round of shuffling. Thus, human antibodies having the desired antigen binding selectivity and affinity may be selected.

Alternatively, a “ribosome display” method may be used that alternates antibody selection with affinity maturation. In the ribosome display method, antibody-encoding nucleic acid is amplified by RT-PCR between the selection steps. Thus, error prone polymerases may be used to introduce mutations into the nucleic acid (see, e.g., Hanes et al., supra).

### Ceruloplasmin Antibody Binding Assays

Antibodies for use in various embodiments of the ceruloplasmin immunoassays disclosed herein may be screened for binding to ceruloplasmin (for example, human, mouse, dog, cat, horse) using certain routine methods that detect binding of an antibody to an antigen. In some embodiments, similar methods and assay formats are used to detect ceruloplasmin levels in the body fluids obtained from patents that are thought to be at some risk for elevated ICP. Such disorders include but are not limited to, TBI or other sources of brain injury, head trauma, polytrauma, stroke, meningitis, encephalitis, hydrocephalus, spinal cord injury, blocked shunts, tumors, for example those of the central nervous system which increase ICP, for example those that cause impingement or occlusion. For example, the ability of a monoclonal antibody to bind ceruloplasmin may be assayed by standard immunoblotting methods, such as electrophoresis and Western blotting (see, e.g., “Current Protocols in Molecular Biology”, supra, Ch. 10.8, and “Antibodies: A Laboratory Manual”, supra). Alternatively, the ability of a monoclonal antibody to bind ceruloplasmin may be assayed using a competitive binding assay, which evaluates the ability of a candidate antibody to compete with a known anti-ceruloplasmin antibody for binding to ceruloplasmin. Competitive binding assays may be performed in various formats including but not limited to ELISA (see, e.g., “Antibodies: A Laboratory Manual”, supra, Ch. 14) the results of which are determined using a colorimeter with one or more fixed wavelengths, or a variable wavelength spectrophotometer, or an ELISA reader, or a fluorometer. In some embodiments, such assays are used to determine ceruloplasmin levels in body fluids obtained from patents that are thought to be at some risk for elevated ICP, to determine whether the patient requires additional monitoring and treatment or requires more intense or frequent monitoring.

A binding assay may be used to quantify the binding kinetics (e.g., rate constant) or the binding affinity (e.g., association or dissociation constant) of an antibody against ceruloplasmin. The binding kinetics or binding affinity can be determined in the “solid-phase” by immobilizing antigen (e.g., ceruloplasmin) on a solid support. In such assays, the immobilized antigen “captures” antibody from solution. Alternatively, binding kinetics or binding affinity may be determined using ELISA-based methods, or using biosensor-based technology, such as Biacore surface plasmon resonance technology (Biacore International AB, Uppsala, Sweden). Many such methods are known to those skilled in the art (see, e.g., “Antibody Engineering: A Practical Approach” (McCafferty et al., eds., Oxford University Press, Oxford, United Kingdom, 1996), Goldberg et al., Curr. Opin. Immunol. 5:278-281, 1993, Karlsson et al., J. Immunol. Methods 145:229-240, 1991, Malmqvist, Curr. Opin. Immunol. 5:282-286, 1993, and Hoogenboom, supra).

The binding kinetics or binding affinity of a Fab fragment that selectively binds to ceruloplasmin may also be determined. Fab fragments do not multimerize. Multimerization may, in certain instances, complicate the measurement of binding kinetics and binding affinity in “solid phase” methods. Thus, Fab fragments that selectively bind to ceruloplasmin may be suitable for use in certain binding assays in which antigen is immobilized to a solid support, such as, for example, an ELISA-based or Biacore assay. Fab fragments may be generated from an intact antibody that selectively binds to ceruloplasmin using enzymatic methods, or by expressing nucleic acids encoding Fab fragments in a recombinant expression system.

Alternatively, the binding kinetics or binding affinity of an antibody against ceruloplasmin can be determined using “solution phase” methods. The measurement of the binding kinetics or the binding affinity of multivalent antibodies and antibodies that multimerize are amenable to solution phase analysis. In such techniques, the kinetics or affinity of binding is measured for an antibody-antigen complex in solution. Such techniques are known to those skilled in the art, including, but not limited to, the “kinetic exclusion assay” (see, e.g., Blake et al., J. Biol. Chem. 271:27677-27685, 1996, and Drake et al., Anal. Biochem. 328:35-43, 2004). Sapidyne Instruments, Incorporated (Boise, Id.), among others, provides instrumentation for performing kinetic exclusion assays. These types of assays may be used to determine ceruloplasmin levels in body fluids obtained from patents that are thought to be at some risk for elevated ICP in some instances.

Monoclonal antibodies raised for example against mouse ceruloplasmin may be screened for selective binding to human, dog, cat or horse ceruloplasmin using routine detection methods, such as those described herein. The ability of a monoclonal antibody to selectively bind both mouse and human ceruloplasmin or those of other mammals (i.e., “cross-reactivity”) indicates the presence of the same epitope in mouse and human ceruloplasmin or other mammal ceruloplasmin. In detection methods that use denaturing conditions (e.g., Western blot), cross-reactivity indicates the monoclonal antibody binds to the same “linear” epitope in mouse and human ceruloplasmin. In detection methods that use non-denaturing conditions, cross-reactivity indicates the monoclonal antibody binds to the same linear epitope or conformational epitope in mouse and human and other mammal ceruloplasmin.

The epitope to which a monoclonal antibody binds may be identified by any of a number of assays (see, e.g., Morris, Methods Mol. Biol. 66:1-9, 1996). For example, epitope mapping may be achieved by gene fragment expression assays or peptide-based assays. In a gene fragment expression assay, for example, nucleic acids encoding fragments of ceruloplasmin are expressed in prokaryotic cells and isolated. The ability of a monoclonal antibody to bind those fragments is assessed, e.g., by immunoblotting or immunoprecipitation. Nucleic acids encoding fragments of ceruloplasmin can be transcribed and translated in vitro in the presence of radioactive amino acids. The radioactively labeled fragments of ceruloplasmin may then tested for binding to a monoclonal antibody. Fragments of ceruloplasmin may also be generated by proteolytic fragmentation. An epitope may also be identified using libraries of random peptides displayed on the surface of phage or yeast, or a library of overlapping synthetic peptide fragments of ceruloplasmin, and testing for binding to a monoclonal antibody. An epitope may also be identified using a competition assay, such as those described below.

Monoclonal antibodies that bind to the same epitope of ceruloplasmin as a monoclonal antibody of interest may be identified by epitope mapping, as described above, or by routine competition assays (see, e.g., “Antibodies: A Laboratory Manual”, supra, Ch. 14, and Morris, supra). In an exemplary competition assay, ceruloplasmin, or a fragment thereof, is immobilized onto the wells of a multi-well plate. The monoclonal antibody of interest is labeled with a fluorescent label (e.g., fluorescein isothiocyanate) by standard methods, and then mixtures of the labeled monoclonal antibody of interest and an unlabeled test monoclonal antibody are added to the wells. The fluorescence in each well is quantified to determine the extent to which the unlabeled test monoclonal antibody blocks the binding of the labeled monoclonal antibody of interest. Monoclonal antibodies may be deemed to share an epitope if each blocks the binding of the other by 50% or greater.

Alternatively, to determine if two or more monoclonal antibodies bind the same epitope, epitope binning may be performed (see, e.g., Jia et al., J. Immunol. Methods 288:91-98, 2004), using, for example, Luminex® 100 multiplex technology and the Luminex® 100™ analyzer (Luminex Corporation, Austin, Tex.). Epitope binning typically utilizes an antibody sandwich-type competition assay, in which a “probe” antibody is tested for binding to an antigen bound by a “reference” antibody. If the probe antibody binds to the same epitope as the reference antibody, it will not bind efficiently to the antigen, because that epitope is masked by the reference antibody. Immunoassays based on the above described technologies and devices (both those named and implied) are employed in various embodiments to detect ceruloplasmin levels in the body fluids from patents that are thought to be at risk for elevated ICP.

Antibodies directed against ceruloplasmin, or conserved variants or peptide fragments thereof, which are discussed above, may also be used in identifying patients at high risk for elevated ICP in diagnostic and/or prognostic assays, as described herein. Such diagnostic and/or prognostic methods may be used to detect abnormalities in the level of ceruloplasmin in a patient's body fluid or tissues and may be performed in vivo or in vitro, such as, for example, on biopsy tissue. For example, antibodies directed to epitopes of ceruloplasmin can be used in vivo to detect the level of ceruloplasmin in the body. Such antibodies can be labeled, e.g., with a radio-opaque or other appropriate compound, and injected into a subject, in order to visualize binding to ceruloplasmin expressed in the body, using methods such as X-rays, CAT-scans, or MRI. In certain embodiments, labeled antibody fragments, e.g., a Fab or single chain antibody comprising the smallest portion of the antigen binding region, may be preferred for this purpose, to promote crossing the blood-brain barrier and permit labeling of ceruloplasmin expressed in the brain.

Alternatively, immunoassays or fusion protein detection assays may be utilized on biopsy and autopsy samples in vitro to permit assessment of the expression pattern of ceruloplasmin. Such assays may include the use of antibodies directed to epitopes of any of the domains of ceruloplasmin. For example, in various embodiments antibodies, or fragments thereof, are used to quantitatively or qualitatively detect ceruloplasmin, conserved variants, or peptide fragments thereof. This may be accomplished, for example, by immunofluorescence techniques employing a fluorescently labeled antibody coupled with ultraviolet microscopic, flow cytometric, or fluorometric detection.

The ceruloplasmin antibodies (or fragments thereof), or ceruloplasmin fusion or conjugated proteins, determining ceruloplasmin levels may, additionally, be employed histologically, for example in immunofluorescence, immunoelectron microscopy, or non-immuno assays, for in situ detection of ceruloplasmin or conserved variants or peptide fragments thereof. In situ detection may be accomplished by removing a histological specimen from a patient, and applying thereto a labeled antibody or fusion protein, performing some embodiments of a ceruloplasmin immunoassay. The antibody (or fragment) or fusion protein is preferably applied by overlaying the labeled antibody (or fragment) onto a biological sample. Through the use of such a procedure, it is possible to determine not only the presence of ceruloplasmin, or conserved variants or peptide fragments, but also its distribution in the examined tissue.

Immunoassays and non-immunoassays for ceruloplasmin, or conserved variants or peptide fragments thereof, will typically comprise incubating a sample, such as a biological fluid, a tissue extract, freshly harvested cells, or lysates of cells that have been incubated in cell culture, in the presence of a detectably labeled antibody capable of identifying ceruloplasmin, or conserved variants or peptide fragments thereof, and detecting the bound antibody by any of a number of techniques well-known in the art. The biological sample may be brought in contact with and immobilized onto a solid phase support or carrier such as nitrocellulose, or other solid support that is capable of immobilizing cells, cell particles, or soluble proteins. The support may then be washed with suitable buffers, followed by treatment with the detectably labeled ceruloplasmin antibody or fusion protein. The solid phase support may then be washed with the buffer a second time to remove unbound antibody or fusion protein. The amount of bound label on solid support may then be detected by conventional means.

The terms “solid phase support” or “carrier” are intended to include any support or carrier capable of binding an antigen or an antibody. Well-known supports or carriers include, but are not limited to, glass, polystyrene, polypropylene, polyethylene, polyvinylidene fluoride, dextran, nylon, amylases, natural and modified celluloses, polyacrylamides, gabbros, and magnetite. The nature of the carrier may be either soluble to some extent or insoluble. The support material may have virtually any possible structural configuration, provided that the coupled molecule is capable of binding to an antigen or antibody. Thus, the support configuration may be spherical, as in a bead, or cylindrical, as in the inside surface of a test tube, or the external surface of a rod. Alternatively, the surface may be flat, such as a sheet or test strip. Preferred supports include polystyrene beads. Those skilled in the art will know many other suitable carriers for binding antibody or antigen, or will be able to ascertain the same by use of routine experimentation.

The binding activity of a given lot of ceruloplasmin antibody or ceruloplasmin fusion protein may be determined according to well-known methods. Those skilled in the art will be able to determine operative and optimal assay conditions for each determination by employing routine experimentation.

With respect to antibodies, one of the ways in which a ceruloplasmin antibody may be detectably labeled is by linking the same to an enzyme for use in an enzyme immunoassay (see, e.g., “Immunoassays: A Practical Approach” (Gosling, ed., Oxford University Press, Oxford, United Kingdom, 2000)). The enzyme that is bound to the antibody will react with an appropriate substrate, preferably a chromogenic substrate, in such a manner as to produce a chemical moiety that may be detected, for example, by spectrophotometric, fluorimetric, or visual means. These assays are read and analyzed using chromatometers, spectrophotmeters and fluorometers, respectively. Enzymes that may be used to detectably label the antibody include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, glucose oxidase, asparaginase, beta-galactosidase, ribonuclease, urease, catalase, glucose-6-phosphate dehydrogenase, glucoamylase and acetylcholinesterase. The detection may be accomplished by colorimetric methods that employ a chromogenic substrate for the enzyme. The detection may also be accomplished using methods that employ a fluorogenic substrate in an enzyme-lined fluorescence (ELF) assay. Detection may also be accomplished by visual comparison of the extent of enzymatic reaction of a substrate in comparison with similarly prepared standards.

Additionally, detection may also be accomplished using any of a variety of other immunoassays. For example, by radioactively labeling ceruloplasmin antibodies or antibody fragments, it is possible to detect ceruloplasmin through the use of a radioimmunoassay (RIA). The radioactive isotope may be detected, for example, by using a gamma or scintillation counter, or by autoradiography. Such antibodies or fragments may also be labeled with a fluorescent compound. When a fluorescently labeled antibody is exposed to light of the proper wavelength, it may be detected due to fluorescence. Exemplary fluorescent labeling compounds include, but are not limited to, fluorescein isothiocyanate, rhodamine, phycoerythrin, phycocyanin, allophycocyanin, o-phthaldehyde, and fluorescamine. Such antibodies may also be detectably labeled using a fluorescence emitting metal, such as ¹⁵²Eu, or others of the lanthanide series. These metals may be attached to an antibody or fragment using such metal chelating groups as diethylenetriaminepentacetic acid (DTPA) or ethylenediaminetetraacetic acid (EDTA).

A ceruloplasmin antibody, or fragment thereof, also may be detectably labeled by coupling it to a chemiluminescent compound. The presence of the chemiluminescent-tagged antibody or fragment is detected by luminescence that arises during the course of a chemical reaction. Examples of useful chemiluminescent labeling compounds include, but are not limited to, luminol, isoluminol, theromatic acridinium ester, imidazole, acridinium salt and oxalate ester. Likewise, a bioluminescent compound may be used to label the ceruloplasmin antibodies, in some cases. Bioluminescence is a type of chemiluminescence found in biological systems, in which a catalytic protein increases the efficiency of the chemiluminescent reaction. The presence of a bioluminescent antibody or fragment is once again detected by luminescence. Exemplary bioluminescent compounds for purposes of labeling include, but are not limited to, luciferin, luciferase and aequorin (green fluorescent protein; see, e.g., U.S. Pat. Nos. 5,491,084, 5,625,048, 5,777,079, 5,795,737, 5,804,387, 5,874,304, 5,968,750, 5,976,796, 6,020,192, 6,027,881, 6,054,321, 6,096,865, 6,146,826, 6,172,188 and 6,265,548).

Immunochromatographic assays, also called lateral flow tests or simply strip tests, are a logical extension of the technology used in latex agglutination tests, the first of which was developed in 1956 by Singer and Plotz (Singer J. M. and Plotz C. M., The latex fixation test. I. Application to the serologic diagnosis of rheumatoid arthritis. Am. J. Med. 21, 888, 1956). The benefits of immunochromatographic tests include: their user-friendly format, short time to get test result, long-term stability over a wide range of climates and they are relatively inexpensive to make. These features make strip tests ideal for applications such as home testing, rapid point of care testing, and testing in the field, in places such as but not limited to the battlefield. In addition, they provide reliable testing that might not otherwise be available to rural environments or third world countries. Thus, such a format would have particular applicability in some cases by facilitating assessment of ceruloplasmin levels at the scene of an accident, on the battlefield, during transport to or rapidly upon arrival at a medical facility.

The principle behind the test is straightforward: any ligand that can be bound to a visually detectable solid support, such as dyed microspheres, may be tested for qualitatively, and in many cases even semi-quantitatively. Some of the more common lateral flow tests currently on the market are tests for pregnancy, Strep throat, and Chlamydia. The two predominant approaches to lateral flow tests are the non-competitive (or direct) and competitive (or competitive inhibition) reaction format. The non-competitive (or direct) double antibody sandwich reaction format is used when testing for larger analytes with multiple antigenic sites, such as, for example, LH, hCG, and HIV. In this format, less than an excess of sample analyte is desired, so that some of the microspheres will not be captured at the capture line, and will continue to flow toward the second line of immobilized antibodies, the control line. This is usually a species-specific anti-immunoglobulin antibodies, specific for the conjugate antibodies on the microspheres. The competitive reaction format is used most often when testing for small molecules with single antigenic determinants, which cannot bind to two antibodies simultaneously. If this format is chosen, it is important to pay close attention to the amount of antibody bound to the microspheres, in relation to the amount of free antigen in the sample. If the sample does not contain an excess of free antigen, some of the microspheres will bind at the capture line, giving a weak signal, and making the test result ambiguous. Typically, the membranes used to hold the antibodies in place are made up of primarily hydrophobic materials. Both the microspheres used as the solid phase supports and the conjugate antibodies are hydrophobic, and their interaction with the membrane allows them to be effectively dried onto the membrane.

One variation to the above reaction formats which avoids the potential problem of protein-coated microspheres sticking to the membrane non-specifically includes using a membrane that is inert and does not bind antibodies. This makes migration of the mobile phase antibodies more efficient and reliable. The capture antibodies, rather than being physically bound by the membrane, are attached to large microspheres, which will be held in place physically, rather than chemically, as the sample passes by, much like boulders in a stream. This may be used for both of the above-mentioned reaction formats. These principles are well-documented in the literature (See for example, U.S. Pat. Nos. 5,141,850, 5,160,701, 5,415,994, 5,451,504, 5,559,041, 5,886,154, 5,925,344, 6,093,804, 6,307,028, 6,913,888, 6,955,917, 7,358,055, 7,361,473, 7,427,490; published PCT Patent Application Nos: WO 88/08534 and WO 91/12336; and published European Patent Application Nos.: 0 284 232 A1 and 0 505 636 A1).

The technology involved in these lateral flow assays, provide an accurate, easy to use, rapid diagnostic tool. Additional approaches include using the same format for lateral flow tests and dyeing the solid support with a fluorescent dye, the possibility exists to create a truly quantitative test. If the spectral properties of the dyed microspheres to which the antibodies are conjugated are known, the amount of antibody bound at the capture line may be precisely quantified using a fluorometer. The benefit to this is that these tests may become truly quantitative assay. In addition, by placing multiple lines of capture antibodies on the membrane, each for a different analyte, one may develop a single test for more than one analyte. For example, this format was used to create a drugs-of-abuse test panels and Biosite's “Triage” was based also on this format (Bangs L. B. (1997) Immunological Applications of Microspheres. The Latex Course Biosite Company, 11030 Roselle St., San Diego, Calif. 92121). Diagnostically, this principle may be used for panels for which multiple analytes are tested, such as immune diseases, allergies, or even Multiple Chemical Sensitivity Disorder.

Lateral flow assay technology is also used in the environmental field, where the format provides a rapid, reliable test that may be performed in the field for anything from water pollution to plant disease. Because these diagnostic tests are often performed in harsh environments, the lateral flow format is ideal. With proper preparation and foil pouching, no refrigeration or special handling is required. It also has use in the field of molecular genetics as a simple format for detecting various genetic markers, and DNA- or RNA-related infectious disease pathogens. The guiding principle behind this type of test, the ability to bind a ligand from solution to a solid support, may be performed on genetic material as well as proteins. Another method that potentially results in reduced development time, includes the use of protein-coated microspheres, such as ProActive® Streptavidin coated microspheres. By biotinylating the desired conjugate antibodies and then taking advantage of the strong affinity that biotin has for streptavidin, the antibodies are easily attached to the microspheres. Alternatively, Protein A or Protein G coated microspheres will bind many IgG's at the Fc region, allowing for optimized, directed antibody attachment. In this way, a series of assays may be developed rather quickly, using the same solid support, membrane, and housing, for example. The only variable would be the conjugate and capture line antibodies used for each specific test. Additional information regarding these microspheres can be found in TechNotes, #51-“ProActive® Protein Coated Microspheres”. By combining several of these approaches a low-cost, rapid quantitative diagnostic assay for multiple analytes may be prepared for use in the field. For examples of use and methods of preparing such assays see, inter alia, U.S. Pat. Nos. 4,435,504, 4,594,327, 4,624,929, 4,756,828, 4,757,004, 4,837,395, 4,857,455, 4,911,794, 4,945,205, 4,956,275, 4,999,285, 5,039,607, 5,075,078, 5,087,556, 5,089,383, 5,164,294, 5,234,813, 5,248,619, 5,252,293, 5,998,221, 5,939,331, 5,908,757, 5,624,809, 5,529,752, 5,468,647, 5,451,507, 5,435,970, 5,334,513, 6,194,220, 6,277,646, 6,686,167, 6,855,561, 6,969,591, 7,067,264, 7,179,657 and 7,226,793; and published U.S. Patent Application Nos.: 20080160549, 20070281370, 20070190531, 20070111323, 20060160084, 20040068101, 20040058395, 20030211634, 20030157699, 20030100128, 20030049167, 20030027222, 20020106696, 20020085958.

In some embodiments of the testing methods described herein, an immunochromatographic assay, or lateral flow test for the level of ceruloplasmin in a sample of a patient's body fluid is used, which provides several potential benefits. For example, in some applications, it may be used in a kit designed to facilitate the process of identifying individuals at increased risk for elevated ICP in rural areas, on the battlefield or even in a physician or veterinarian's office. Such an assay need be only semi-quantitative, as only those samples in which serum ceruloplasmin levels are decreased below the normal range need be detected. As an immunoassay, this test may be titrated to obtain the desired cutoff and sensitivity. Such an assay may also be combined with a colorimetric assay to simultaneously detect copper levels in the same test sample. Alternatively, an immunoassay for copper may be obtained and used in combination with a ceruloplasmin immunoassay in the same test sample. As described above, in some embodiments, levels of copper and/or ceruloplasmin in a body fluid other than serum (e.g., plasma, blood, cerebral spinal fluid, urine and saliva) are used as surrogate markers indicative of serum levels, wherein a subnormal level of serum copper or serum ceruloplasmin, or both, indicates increased risk for elevated intracranial pressure.

Nephelometry is a technique performed by shining light on a sample, and measuring the amount of light scattered. This technique is widely used in clinical laboratories because it is relatively easily automated. It is based on the principle that a dilute suspension of small particles will scatter light (often but not necessarily a laser) passed through it rather than simply absorbing it. The amount of scatter is determined by collecting the light at an angle (usually at about 70 or 75 degrees). Such immunoturbidimetric assays (protein immunoassays) are read using a Photometer (such as a DTN-410, DIALAB GmbH, Germany) or autolyzer Photometer (DTN-410K, DIALAB GmbH). Antibody and the antigen are mixed in concentrations such that only small aggregates are formed that do not quickly settle to the bottom. The amount of light scatter is measured and compared to the amount of scatter from known mixtures. The amount of the unknown is determined from a standard curve. Enzyme multiplied immunoassay technique, or EMIT, is a common method for screening urine and blood for drugs, both legal and illicit. First introduced by Syva Company in 1973, it is the first homogeneous immunoassay to be widely used commercially. A mix and read protocol has been developed that is exceptionally simple and rapid. The most widely used applications for EMIT are for therapeutic drug monitoring (serum) and as a primary screen for abused drugs and their metabolites (urine). The early U.S. patents covering the major aspects of the method, Nos. 3,817,837 and 3,875,011, are expired. While still sold by Siemens Healthcare under its original tradename, EMIT, assay kits with different names that employ the same technology are supplied by other companies. The method is highly reliable and reliance on its results has even been upheld by the US Supreme Court. Older ELISAs utilize chromogenic substrates, while many of the newer assays employ fluorogenic substrates enabling increased sensitivity, both types can be read on an ELISA microplate reader (such as the DIAREADER:DIALAB GmbH, Germany) of the appropriate measuring range, for example from 400 nm to 750 nm.

Additionally, analytical scale immunoprecipitations may also be used to detect the presence of an analyte, such as ceruloplasmin in body fluids. Monodispersed magnetic beads are also available as a support material which offers certain advantages over polydisperse agarose beads. Magnetic beads have the ability to bind extremely large protein complexes and the complete lack of an upper size limit for such complexes, as unlike agarose beads which are sponge-like porous particles of variable size, magnetic beads are small, solid and (in the case of monodisperse magnetic beads) spherical and uniform in size. The lower overall binding capacity of magnetic beads for immunoprecipitation make it much easier to match the quantity of antibody needed for diagnostic immunoprecipitations precisely with the total available binding capacity on the beads which results in decreased background and fewer false positives. The increased reaction speed of the immunoprecipitations using magnetic bead technologies results in superior results when the analyte protein is labile due to the reduction in protocol times and sample handling requirements which reduces physical stresses on the samples and reduces the time that the sample is exposed to potentially damaging proteases. Agarose bead-based immunoprecipitations may also be performed more quickly using small spin columns to contain the agarose resin and quickly remove unbound sample or wash solution with a brief centrifugation (Celis, J. E., Lauridsen, J. B., and Basse, B. (1994) Determination of antibody specificity by Western blotting and immunoprecipitation. In: Celis, J. E. (ed.), CELL BIOLOGY. A LABORATORY HANDBOOK, Academic Press, New York, Vol. 2, pp. 305-313. Mason, D. W., and Williams, A. F. (1986) Kinetics of antibody reactions and the analysis of cell surface antigens. In: Weir, D. M., Herzenberg, L. A., Blackwell, C., and Herzenberg, L. A. (ed.), HANDBOOK OF EXPERIMENTAL IMMUNOLOGY, Blackwell, Oxford, vol. 1, chapter 38.

### Detection of Increased Risk of Elevated ICP

In various applications, a testing method is employed for diagnostic and/or prognostic evaluation of patients who might be at risk for elevated ICP during disorders associated with elevated ICP. Elevated ICP can result as a consequence of TBI or other related disorders which elevate ICP, such as, but not limited to, brain injury, head trauma, polytrauma, stroke, meningitis, encephalitis, hydrocephalus, spinal cord injury, blocked shunts, tumors, for example those of the central nervous system (CNS) which increase ICP, for example those that cause impingement or occlusion.

Ceruloplasmin levels may be measured by assaying its enzymatic activity, or through the use of immunoassays, in many different formats including, but are not limited to, radioimmunoassay (RIA), electroimmunoassay, enzyme-linked immunoassay systems (ELISA), immunonephelometry or immunoturbidimetry, immunochromatography, immunoprecipitation, immunofluormetry, as well as those immunoassays that are less often used for high throughput, such as, but not limited to Western blots, immunodiffusion, immunoelectrophoresis, immunohistochemistry. Thus, it can be appreciated that a wide variety of ceruloplasmin measurement technologies are currently available to implement various embodiments of the disclosed methods to identify patients at increased risk of developing elevated ICP by determining decreased levels of serum ceruloplasmin and/or serum copper indicative of a pending rise in ICP. Alternatively, any other suitable technology for measuring, estimating or predicting ceruloplasmin levels may be used in various embodiments of these methods.

### Detection of Ceruloplasmin

One exemplary testing method is performed by utilizing pre-packaged diagnostic kits comprising a ceruloplasmin nucleotide sequence, a ceruloplasmin protein or peptide, and/or a ceruloplasmin antibody reagent described herein, which may be conveniently used, e.g., in clinical settings, to identify patients who will develop elevated ICP. Any suitable clinical assay currently used or approved for use in determining human or animal ceruloplasmin levels in serum or in another body fluid, such as but not limited to plasma, blood, urine, cerebral spinal fluid (CSF), urine and saliva can be used in various embodiments of the disclosed methods, and correlated to serum levels. Such assays that are currently used to diagnose and follow Wilson's disease patients can be used in the present method to identify individuals at risk for elevated ICP. Some of these assays for ceruloplasmin and copper are described in, among other things, the diagnosis and treatment of Wilson's disease, in U.S. Pat. Nos. 4,404,132; 4,722,894; 4,886,780; 5,108,987; 5,491,066; 6,372,262; 6,780,842; 6,783,775; 6,806,044; 7,368,190; 7,407,743. Ceruloplasmin and copper assays which may be used also include those described in, among others, published U.S. patent application document nos.: US20020054916, US20020094949, US20020110523, US20020142360, US20020094949, US20030054980, US20030054980, US20040214327, US20050042693, US20070161120, US20080044721, US20080118782, US20080286868; in published PCT applications nos.: WO/1985/000812, WO/1995/028956, WO/1998/025954, WO/1998/010293, WO/1998/025634, WO/1999/067638, WO/2000/067781, WO/2000/067782, WO/2001/082954, WO/2001/016294, WO/2002/034920, WO/2003/106966, WO/2006/028321; and European Patent Application Nos.: EP0003916, EP0134320, EP0606516, EP0931263, EP0973530, EP1023089, EP1176977, EP1181049; as well as in Japanese Patent Document Nos.: JP02257896, JP03287685, JP04210591, JP05268987, JP06265545, JP08075743, JP10239311, JP51118821, JP2673619, JP56002916, JP56090015, JP56120622, JP57068797, JP58015923, JP58141782, JP60149529, JP60162957, JP61076422, JP61087629, JP61219397, JP61224999, JP62155299, JP63245696 and JP2004196816.

Ceruloplasmin levels may also be measured by assaying its enzymatic (oxidase) activity using any suitable method. One example is a functional assay that relies generally on the ability of ceruloplasmin to catalyze the oxidation of phenylenediamine producing a purple product (e.g., Ravin, H. A. Rapid test for hepatolenticular degeneration, Lancet 1: 726-727, 1956; Cox, D. W. Factors influencing serum ceruloplasmin levels in normal individuals, J Lab Clin Med 68: 893-904, 1966; Walshe, J. M. Wilson's disease: the importance of measuring serum caeruloplasmin non-immunologically, Ann Clin Biochem 40,115-121, 2003 and MacIntyrea, G., Gutfreundb, K. S., Martine, W., Camiciolic, R, Coxa, D. W., Value of an enzymatic assay for the determination of serum ceruloplasmin. J of Lab and Clin Medicine, 144: 294-301, 2004). The rate of this reaction is measured in a spectrophotometric assay with modification. To carry out such an assay 25 μL of serum or EDTA-treated plasma is mixed with 0.67 mL of sodium acetate buffer (1 mol/L, pH 5.2) and 0.33 mL of paraphenylenediamine solution (0.4% paraphenylenediamine dihydrochloride in 1 mol/L freshly made sodium acetate buffer). Substrate oxidation is measured at 35° C. by taking readings at an optical density of 610 nm at 30-second intervals for 5 minutes using a spectrophotometer. The rate of oxidation is calculated as the change in optical density at 610 nm over the 5 minutes. These values are compared to a sample containing a known concentration of ceruloplasmin that is used to produce a standard curve.

Ceruloplasmin may also been determined using immunoassays done using many different formats, such as but not limited to radial immunodiffusion (Matsuda, I., Pearson, T., Holtzman, N. A., Determination of apoceruloplasmin by radioimmunoassay in nutritional copper deficiency, Menkes' kinky hair syndrome, Wilson's disease, and umbilical cord blood, Pediatr Res 8: 821-884, 1974; Cauza, E., Maier-Dobersberger, T., Polli, C., Kaserer, K., Kramer, L. and Ferenci, P. Screening for Wilson's disease in patients with liver diseases by serum ceruloplasmin, J Hepatol, 27: 358-362, 1997), radioimmunoassay (RIA), enzyme-linked immunoassay systems (ELISA), immunonephelometry or immunoturbidimetry, immunochromatography, as well as those immunoassays that are less often used for high throughput, such as, but not limited to western blots, immunodiffusion, immunoelectrophoresis, immunohistochemistry and those described throughout this specification, in the examples below, and known to those of skill in the art.

Yet another example of a technique by which total ceruloplasmin in a sample may be measured is with the use of nephelometry and electroimmunoassay (Laurell C. B. “Electroimmuno Assay”. Scand J Clin Lab Invest, 29, Issue 5124, 21-37, 1972) using appropriately diluted serum and EDTA-treated plasma samples and a polyclonal antibody specific for ceruloplasmin (Dako, Burlington, Ontario, Canada). A dilution series derived from a serum sample of known ceruloplasmin concentration, although enzymatically inactive, is subjected to electrophoresis on each gel and used to produce a standard curve (College of American Pathologists, Reference Proteins in Human Serum, Northfield, Ill.). Peak heights for the test samples were applied to the standard curve and their ceruloplasmin concentrations calculated. The activity of the enzyme is assumed to be 100%, an overestimate resulting from the presence of apoCp and unknown inactivation during processing. A conversion factor is subsequently obtained from the standard curve and used to convert the oxidation rates for the test samples to a ceruloplasmin concentration in milligrams per liter (MacIntyrea, G., Gutfreundb, K. S., Martinc, W., Camiciolic, R, Coxa, D. W., Value of an enzymatic assay for the determination of serum ceruloplasmin. J of Lab and Clin Medicine, 144: 294-301, 2004).

An additional assay for ceruloplasmin in human serum and plasma has been described for use on Olympus analysers The CE marked product, OSR 6164, has linearity from 60 to 2000 mg/l of ceruloplasmin. Calibrator and control sera materials are available and come in the kits; this assay exhibits good hemolysate, bilirubin, and lipaemic interference characteristics.

Additional ceruloplasmin assays are described in the literature, including: Gutteridge J M C, et al. Ceruloplasmin: physiological and pathological perspectives. CRC Crit. Rev Clin Lab Sci; 14:257-329, 1981; Schaefer M and Gitlin J D. Genetic disorders of membrane transport. IV. Wilson's disease and Menkes disease. Am J Physiol 1999 February; 276(2 Pt 1): G311-314. Vanhoutte, P. M., Endothelial dysfunction and atherosclerosis. Eur Heart J 18 (Suppi. E): E19-E39, 1997; Ehrenwald E. Chisolm G M, Fox P L. Intact human ceruloplasmin oxidatively modifies low density lipoprotein. J Clin Invest 93: 1493-1501, 1994. Deiss A. Wilson's disease. In: CECIL TEXTBOOK OF MEDICINE, Wyngaarden J B, Smith L H, Bennett J C (eds). Vol 1, 19th Edition. W.B. Saunders Co., Philadelphia, Pa.; pp: 1378-1527, 1992. Gahl W A. Wilson's disease. In: CECIL TEXTBOOK OF MEDICINE, 21st ed. Goldman L and Bennett J C (eds). W.B. Saunders Co., Philadelphia, Pa. 1130-2, 2000. Hoffman H N II. Phyliky R. L. and Fleming, C. R. Zinc-induced Copper Deficiency. Gastroenterology, 1988; 94(2): 508-12. Cauza E. Maier-Dobersberger T. Polli C et. al. Screening for Wilson's Disease on Patients with Liver Diseases by Serum Ceruloplasmin. J Hepatol, 27(2): 358-62, 1997.

Still further, many of the published assays originally developed and used to determine ceruloplasmin levels in body fluids to facilitate screening diagnosis or studies related to Wilson's disease or Menke's syndrome may also be used in some embodiments of the methods disclosed herein, to determine serum ceruloplasmin or serum copper levels to indicate whether an individual is at risk for elevated ICP. Such assays are presented in, but not limited to Yarze, J. C., Martin P., Munoz, S. J., Friedman, L. S., Wilson's Disease: current status. Am J Med 92:643-54, 1992; K. Terada, Y. Kawarada, N. Miura, Yasui, O., Koyama, K. and T. Sugiyama, Copper incorporation into ceruloplasmin in rat livers, Biochim Biophys Acta 1270:58-62, 1995; E. Cauza, T. Maier-Dobersberger, C. Polli, K. Kaserer, L. Kramer and P. Ferenci, Screening for Wilson's disease in patients with liver diseases by serum ceruloplasmin, J Hepatol 27: 358-362, 1997; Z. L. Harris, Y. Takahashi, H. Miyajima, M. Serizawa, R. T. MacGillivray and J. D. Gitlin, Aceruloplasminemia: molecular characterization of this disorder of iron metabolism, Proc Natl Acad Sci USA 92: 2539-2543, 1995; K. Yoshida, K. Furihata, S. Takeda, A. Nakamura, K. Yamamoto and H. Morita et al., A mutation in the ceruloplasmin gene is associated with systemic hemosiderosis in humans, Nat Genet. 9: 267-272, 1995; K. S. Raju, G. Alessandri, M. Ziche and P. M. Gullino, Ceruloplasmin, copper ions, and angiogenesis, J Natl Cancer Inst 69: 1183-1188, 1982; K. Gibbs and J. M. Walshe, A study of the caeruloplasmin concentrations found in 75 patients with Wilson's disease, their kinships and various control groups, Q J Med 48: 447-463, 1979; N. Kojimahara, H. Nakabayashi, T. Shikata and M. Esumi, Defective copper binding to apo-ceruloplasmin in a rat model and patients with Wilson's disease, Liver 15: 135-142, 1995; E. A. Roberts and D. W. Cox, Wilson disease, Baillieres Clin Gastroenterol 12: 237-256, 1998; K. Gibbs and J. M. Walshe, A study of the caeruloplasmin concentrations found in 75 patients with Wilson's disease, their kinships and various control groups, Q J Med 48: 447-463, 1979; I. H. Scheinberg and I. Sternlieb, WILSON'S DISEASE, vol XXIII, Saunders, Philadelphia (1984). I. Sternlieb and I. H. Scheinberg, The role of radiocopper in the diagnosis of Wilson's disease, Gastroenterology 77: pp. 138-142, 1979; J. F. Kurtzke, Normal ceruloplasmin in Wilson's disease, Arch Neurol 7: 371-376, 1962; A. G. Rosenoer, Ceruloplasmin in Wilson's disease, Lancet 2:1163-1164, 1959; P. Steindl, P. Ferenci, H. P. Dienes, G. Grimm, I. Pabinger and C. Madl et al., Wilson's disease in patients presenting with liver disease: a diagnostic challenge, Gastroenterology 113: 212-218, 1997; A. Yuce, N. Kocak, H. Ozen and F. Gurakan, Wilson's disease patients with normal ceruloplasmin levels, Turk J Pediatr 41: 99-102, 1999; P. J. Gow, R. A. Smallwood, P. W. Angus, A. L. Smith, A. J. Wall and R. B. Sewell, Diagnosis of Wilson's disease: an experience over three decades, Gut 2000 46: 415-419, 2000; J. M. Walshe, Wilson's disease: the importance of measuring serum caeruloplasmin non-immunologically, Ann Clin Biochem 40: 115-121, 2003; M. O. Louro, J. C. Tutor and J. M. Paz, Serum and plasma ceruloplasmin in humans, J Clin Chem Clin Biochem 27: 511-513, 1989; D. W. Cox, Factors influencing serum ceruloplasmin levels in normal individuals, J Lab Clin Med 68: 893-904, 1966; S. O'Reilly, Observations on ceruloplasmin and methods for its estimation, Neurology 11: 259-265, 1961; C. B. Laurell, Electroimmuno assay, Scand J Clin Lab Invest Suppl 124: 21-37, 1972; H. A. Ravin, Rapid test for hepatolenticular degeneration, Lancet 1: 726-727, 1956; R. Beetham, P. White, P. Riches, D. Bullock and F. MacKenzie, Use of CRM 470/RPPHS has not achieved true consensus for ceruloplasmin measurement, Clin Chem 48: 2293-2294, 2002; M. L. Schilsky and B. Shneider, Population screening for Wilson's disease, J Pediatr 140: 499-501, 2002; S. H. Hahn, S. Y. Lee, Y. J. Jang, S. N. Kim, H. C. Shin and S. Y. Park et al., Pilot study of mass screening for Wilson's disease in Korea, Mol Genet Metab 76: 133-136, 2002.

### Copper Assays

Copper (II) or Cu²⁺ may be assayed in many ways. For example, after preparing a solution of the substance one may add dilute sodium hydroxide solution to the solution and when copper is present a pale blue precipitate that dissolves as more sodium hydroxide is added. Similarly one may add dilute ammonia solution to a sample to be tested, and in the presence of copper a pale blue precipitate will form that will change to deep blue solution as more ammonia is added. The presence of copper has also been confirmed by the use of a flame test. After dipping a metal probe into the solution to be tested and place it in a flame the presence of a green flame rising from the probe signifies the presence of the element copper, a red flame the element calcium and an orange flame the element sodium.

Spectroscopic and spectrophotometric based assays to determine the copper levels present in body fluids are known to those of skill in the art and have been described, often in relation to Wilson's disease. Such assays including the use of atomic absorption spectroscopy are described in the literature. For example, in Stuerenburg, H. J. and Eggers, C., 2000 (Early detection of noncompliance in Wilson's disease by consecutive copper determination in cerebrospinal fluid. J Neurol Neurosurg Psychiatry 69: 701-702); Liska, S. K., Kerkay, J, and Pearson, K. H., 1985 (Determination of zinc and copper in urine using Zeeman effect flame atomic absorption spectroscopy. Clin Chim Acta. 151:231-236); Tessman, R. K, Lakritz J, Tyler, J. W. Casteel, S. W. Williams, J. E., Dew, R. K., 2001 (Sensitivity and specificity of serum copper determination for detection of copper deficiency in feeder calves. J Am Vet Med Assoc. 218:756-760); Vilanova A, Gutiérrez C, Serrat N, et al. 1997 (Metallothionein, zinc and copper levels: relationship with acute myocardial infarction. Clin Biochem 30:235-8); Vulpe C D, Packman S. Cellular copper transport. Annu Rev Nutr 15:293-322, 1995; Gaffney D, Fell G S, and O'Reilly D S, 2000 (Wilson's disease: acute and presymptomatic laboratory diagnosis and monitoring. J Clin Pathol 53:807-12); Bull, P. C., Thomas, G. R., Rommens, J. M., et al., 1993 (The Wilson disease gene is a putative copper transporting P-type ATPase similar to the Menkes gene [correction appears in Nat Genet. 6:214, 1994] Nat Genet; 5:327-37, 1993); Milne, D B, 1994 (Assessment of copper nutritional status. Clin Chem 40:1479-84, 1994); and Dufour, D R, Lott, J A, Nolte, F S, et al. 2000 (Diagnosis and monitoring of hepatic injury. II. Recommendations for use of laboratory tests in screening, diagnosis, and monitoring. Clin Chem 46:2050-68, 2000).

In addition to the copper assay described in Example 1.7 below, another example of a copper assay includes the QuantiChrom™ Copper Assay Kit (DICU-250, BioAssay Systems, Hayward, Calif.) a simple, direct and automation-ready procedure for measuring copper concentrations find wide applications in research, drug discovery and environmental monitoring. This assay kit is designed to measure copper directly in serum or plasma without any pretreatment. The improved method utilizes a chromogen that forms a purple colored complex specifically with copper ions. The intensity of the color, measured at 359 nm, using a spectrophotometer is directly proportional to copper concentration in the sample. The optimized formulation substantially reduces interference by substances in the raw samples. The manufacturer states that this assay also works well with non human samples, for example, rat plasma, rat serum, goat serum and fetal bovine serum (Invitrogen) were assayed using this 96-well plate assay, the copper concentrations were 103±1 (n=4), 106±1 (n=4), 152±2, 114±1 μg/dL (n=4), respectively. CV<2%.

Additional examples of assays that may be used in some embodiments to determine copper concentration in body fluids (e.g., serum, plasma, blood, urine, cerebral spinal fluid (CSF), urine and saliva), includes those that are known or used in clinical laboratories or research laboratories for measuring copper. One such assay is inductively coupled plasma/mass spectrometry. To obtain a specimen for use in this assay one 6 mL royal blue (no additive), or royal blue EDTA tube of serum is collected from the patient. The tube is spun in a centrifuge and the serum or plasma is immediately poured off into an ARUP Trace Element-Free Transport Tube. It is critical that the serum or plasma is not allowed to remain on the cells or clot. A specimen containing 0.5 mL-2 mL of serum or plasma at 20-25° C. is submitted in an ARUP Trace Element-Free Transport Tube (ARUP supply #43116). If the specimen is drawn and stored in the appropriate container, the trace element values will not change with time. Normal reference Intervals using this method are for patients ages 0 up to 6 months: males 20-70 μg/dL, females 20-70 μg/dL; for patients ages 7 months-18 years: males 90-190 μg/dL, females 90-190 μg/dL; for patients ages 19 years and older: males 70-140 μg/dL, females 80-155 μg/dL.

Copper assays are also described in the literature, including, for example, U.S. Pat. No. 5,173,431, and Stuerenburg, H. J., Eggers, C. “Early detection of noncompliance in Wilson's disease by consecutive copper determination in cerebrospinal fluid.” J Neurol Neurosurg Psychiatry 69: 701-702, 2000; Liska, S. K., Kerkay, J., Pearson K H (1985)” Determination of zinc and copper in urine using Zeeman effect flame atomic absorption spectroscopy.” Clin Chim Acta. 151:231-236, 1985; Tessman, R. K., Lakritz, J., Tyler, J. W., Casteel, S. W., Williams, J. E., Dew, R. K., “Sensitivity and specificity of serum copper determination for detection of copper deficiency in feeder calves.” J Am Vet Med Assoc. 218:756-760, 2001. A 24-hour urine copper assay (ARUP's Laboratory Test Directory) is approved for use in humans and is available for clinical use.

### Combination of Copper and Ceruloplasmin Assays

In some cases, a patient suspected of being at some risk for development of elevated ICP has a sample of body fluid drawn and that fluid, or aliquots thereof, are subject to both copper and ceruloplasmin assays. As described above, in some embodiments, levels of copper and/or ceruloplasmin in a body fluid other than serum (e.g., plasma, blood, cerebral spinal fluid, urine and saliva) are used as surrogate markers indicative of serum levels, wherein a subnormal level of serum copper or serum ceruloplasmin, or both, indicates increased risk for elevated intracranial pressure.

Should one or both of those assays reveal that the patient's serum copper or ceruloplasmin levels are below the normal range, then it is assumed that the patient will develop elevated ICP. One approved and clinically available combination of assays is provided by ARUP's Laboratory Test Directory 0020598: Wilson Disease Screening Panel, Serum, which utilizes Immunoturbidimetric/Inductively Coupled Plasma/Mass Spectrometry to determine a patient ceruloplasmin and copper levels respectively. In this particular clinical test (0020096) for copper levels in patient serum, the normal reference interval: for patients from 0 up to 6 months of age, 20-70 μg/dL for males and 20-70 μg/dL for females; for patients from 7 months-18 years of age, 90-190 μg/dL for males and 90-190 μg/dL; for females; and for patients 19 years of age or older, 70-140 μg/dL for males and 80-155 μg/dL for females. The ceruloplasmin aspect of this panel utilizes an immunoturbidimetric assay (0050160) to determine ceruloplasmin levels in patient serum, in this assay the normal reference interval: for patients from 0-17 years of age is 20-43 mg/dL and for patients 18 years of age and older it is 17-54 mg/dL.

In certain embodiments, a method for detecting ceruloplasmin in a sample is provided which comprises contacting sample proteins or peptides from a sample suspected of containing ceruloplasmin with at least a first antibody that binds to a ceruloplasmin protein or peptide, under conditions effective to allow the formation of immune complexes, and detecting the immune complexes thus formed. In some cases, the sample proteins contacted are located within a cell, while in others, the sample proteins are separated from a cell prior to contact. Thus, in some embodiments an immunoassay detection kit is provided that comprises at least a first antibody (e.g., a monoclonal antibody) capable of binding to a ceruloplasmin protein or peptide and a detection reagent, along with testing instructions. In some instances, the immunoassay detection kit also includes an unrelated protein or peptide for use as a control, and/or a second antibody that binds to the first antibody. In some embodiments, the kit contains the necessary components for testing a body fluid for copper level, to determine whether the individual is at increased risk of elevated ICP. Test kits are further described below.

### EXAMPLES

### 1.1 Patient Recruitment and Sample Collection

All protocols regarding the use of human subjects were reviewed and approved by the University of Texas Health Science Center at Houston Committee for the Protection of Human Subjects. Non-trauma volunteers were consented and enrolled by the University of Texas Clinical Research Unit at Memorial Hermann Hospital. TBI patients (14-65 years old) with Glasgow Coma Scale (GCS) scores≦8 were recruited for the study (Teasdale, G. and Jennett, B. 1974. Assessment of coma and impaired consciousness. A practical scale. Lancet 2, 81-84). Brain trauma patients (or their next-of-kin) admitted to the adult neurotrauma intensive care unit (NTICU) of Memorial Hermann Hospital (Houston, Tex.) were consented for participation in this study. ICP measurements were recorded continuously from the time of monitor placement. Elevated ICP was defined as a ≧25 mm Hg measurement for at least 5 minutes that occurred either twice in a 24 hr period, or on two consecutive days. Blood samples were obtained specifically for the purpose of this study and were coded to protect anonymity. An initial blood draw was obtained at the earliest possible time after admission, patient stabilization, and informed consent was obtained. To minimize interference with clinical management, subsequent blood samples were drawn after morning rounds were completed (i.e., every 24 hours). Relevant medical data was recorded and coded to match the extracted blood sample. One 10-ml blood sample was collected from each enrolled subject via existing catheter (in order of preference: arterial, central venous, or peripheral) or by venipuncture of one of the superficial forearm veins. For the brain-injured patients, blood was collected once daily for the first five days post-injury. Serum was isolated by centrifugation at 4° C. using serum separator tubes (Becton Dickinson, Franklin Lakes, N.J.) as described by the vendor. Aliquots were prepared and frozen at −80° C. until needed.

### 1.2 Patient Assessment and Classification

Patients enrolled in this study (14-65 years of age) were classified according to their injuries using initial computed tomography (CT) scans (Hergenroeder, G., Redell, J. B., Moore, A. N., et al. 2008. Identification of serum biomarkers in brain-injured adults: potential for predicting elevated intracranial pressure. J. Neurotrauma 25, 79-93). Data were collected on several physiological scoring systems to stratify patient groups and the level of treatment they received: Acute Physiology and Chronic Health Evaluation II (APACHE II), GCS, Injury Severity Score (ISS), and Therapeutic Intensity Level (TIL). APACHE II is designed to measure the severity of disease for newly admitted ICU patients (Knaus, W. A., Draper, E. A., Wagner, D. P., and Zimmerman, J. E. 1985. APACHE II: a severity of disease classification system. Crit Care Med. 13, 818-829). To calculate the Apache II score, the worst value of each component in the first 24 hours of ICU admission was collected. GCS is one component of the Apache II.

GCS is a neurological assessment routinely used in clinical care and neurosurgical research (Teasdale, et al., 1974, supra). All injuries were classified according to body region and given an abbreviated injury score (AIS, The Abbreviated Injury Scale, 1990 Revision, Update 98) from 1 to 6 according to standardized definitions. The worst three MS scores in different body regions were squared and summed to calculate the ISS. The ISS correlates linearly with mortality, morbidity, length of hospital stay, and other measures of injury severity. Polytrauma was defined as an MS score≧3 in a body region other than the head, neck or face.

### 1.3 Detection of Ceruloplasmin Fragments in Serum

Ceruloplasmin levels were determined in pooled serum samples obtained at either 2 days (41.5±4.9 hr) or 3 days (66.3±6.6 hr) post-TBI was using isobaric tag for relative and absolute quantitation (iTRAQ)-labeled serum samples and analysis as described in Hergenroeder, et al. (2008, supra) “The use of liquid chromatography-mass spectrometry/mass spectrometry (LC-MS/MS) was used to detect 4 ceruloplasmin-derived peptides which were identified using the SwissProt fasta database The levels of these four peptides were significantly increased in serum samples obtained from TBI patients on day 2 (41.5±4.9 hr) or day 3 (66.3±6.6 hr) post-injury.

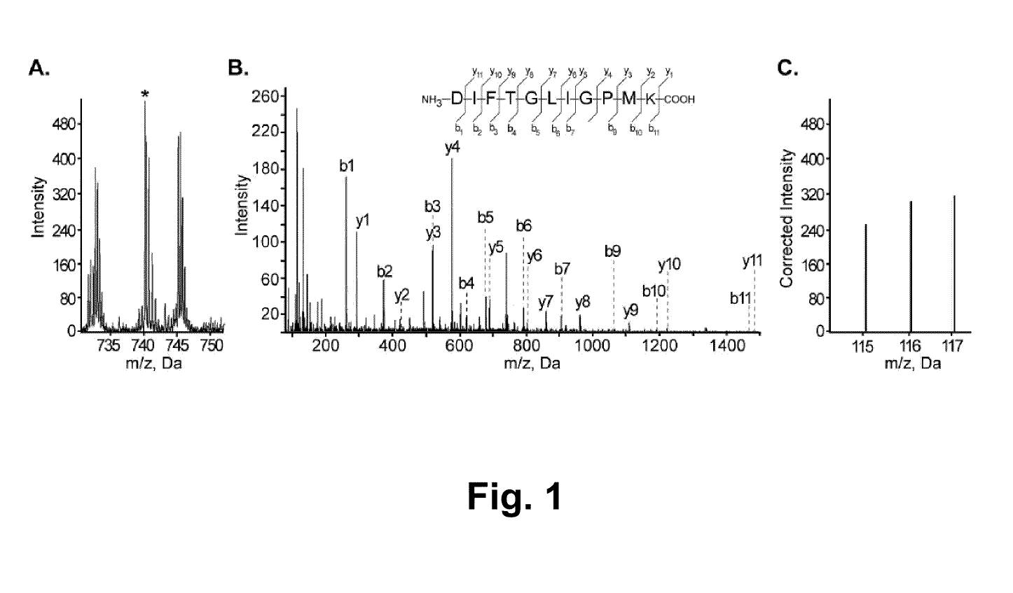

FIG. 1A shows a representative mass spectrometry profile from a ceruloplasmin-derived peptide (m/z ratio of 741, indicated by *). This peak was analyzed in MS/MS mode to obtain the amino acid sequence, further corroborating that the peptide fragment was from the ceruloplasmin protein (FIG. 1B). The intensity of the respective isobaric tag signals for this peptide, from healthy volunteer (115 Da) and TBI samples (2 day: 116 Da; 3 day: 117 Da) are shown in FIG. 1C. These intensities, combined with the intensities recorded for the other ceruloplasmin-derived peptides, were used to calculate the fold change in ceruloplasmin after injury (2 day post-TBI/healthy volunteer: 1.347; 3 day post-TBI/healthy volunteer: 1.597).

### 1.4 Detection of Ceruloplasminin Levels in Serum by ELISA

Using a new cohort of serum samples obtained from healthy volunteers and TBI patients, an ELISA based method was used to determine serum ceruloplasmin levels. Ceruloplasmin levels were measured using a sandwich ELISA kit (GenWay, San Diego, Calif.). A standard curve for calibrating the abundance of ceruloplasmin was generated by serial dilution of purified recombinant ceruloplasmin protein. The range of the standards was based on the manufacturer's instructions. Standards and diluted serum samples were added to a 96-well plate containing immobilized antibodies specific for ceruloplasmin. Samples were incubated at room temperature for 1 hr. Following extensive washing, bound serum proteins were detected using an HRP-conjugated polyclonal antibody against the ceruloplasmin protein. The antibody was incubated for 2 hr at room temperature and detected using 2,2′-Azino-bis-(3-ethylbenzthiazoline-6-sulfonic acid) and 3% H₂O₂. The optical density was measured using a microplate reader at 450 nm.

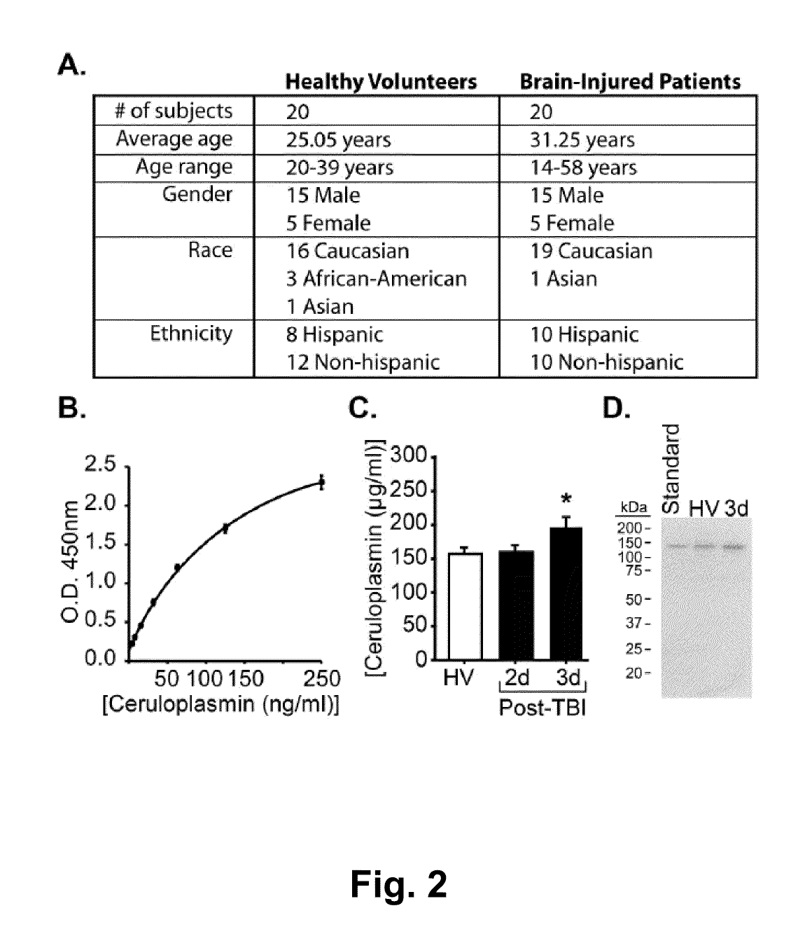

FIG. 2A shows the demographics for the healthy volunteer and TBI groups used for ELISA analysis. FIG. 2B depicts a representative standard curve for ceruloplasmin using purified protein as the standard. Based on this curve, serum samples were diluted accordingly to ensure their concentrations were within the detectable range. The ELISA results shown in FIG. 2C indicate that serum ceruloplasmin levels significantly increase as a function of time compared to the healthy volunteers (one-way ANOVA: F_{(2,53)}=4.02, p=0.024). Post-hoc analysis revealed that this difference was due an increase in serum ceruloplasmin at the 3 day, but not the 2 day, post-injury time point (*, FIG. 2C).

These results could have resulted from either an increase in full-length ceruloplasmin protein, or an increase in ceruloplasmin break-down fragments. In order to distinguish between these possibilities, western blots were done using the detection antibody from the ELISA kit. FIG. 2D shows a representative picture of a western blot using pooled samples from 10 healthy volunteers and 10 TBI patients whose blood was drawn 3 days after their injury. The results indicate the detection antibody only identified a 127 kDa protein that co-migrated with the full length purified protein used to generate the standard curve.

Statistical Analysis: ELISA data was evaluated by a Kolmogorov-Smirnov normality test, and data that did not have a normal distribution was statistically evaluated by a Kruskal-Walis one-way analysis of variance on ranks followed by post-hoc pair-wise comparisons using Dunn's method for multiple comparisons. All other data was compared using either one-way or two-way ANOVAs followed by post-hoc analysis. The diagnostic value of the changes was determined by Receiver Operating Characteristic (ROC) curves (Zweig, M. H. and Campbell, G. (1993). Receiver-operating characteristic (ROC) plots: a fundamental evaluation tool in clinical medicine. Clin. Chem. 39, 561-577). Data are presented as the mean±SEM unless otherwise indicated, and the median value and data range are provided where appropriate.

### 1.5 Patients with ICP≧25 mm Hg have a Significant Decrease in Serum Ceruloplasmin

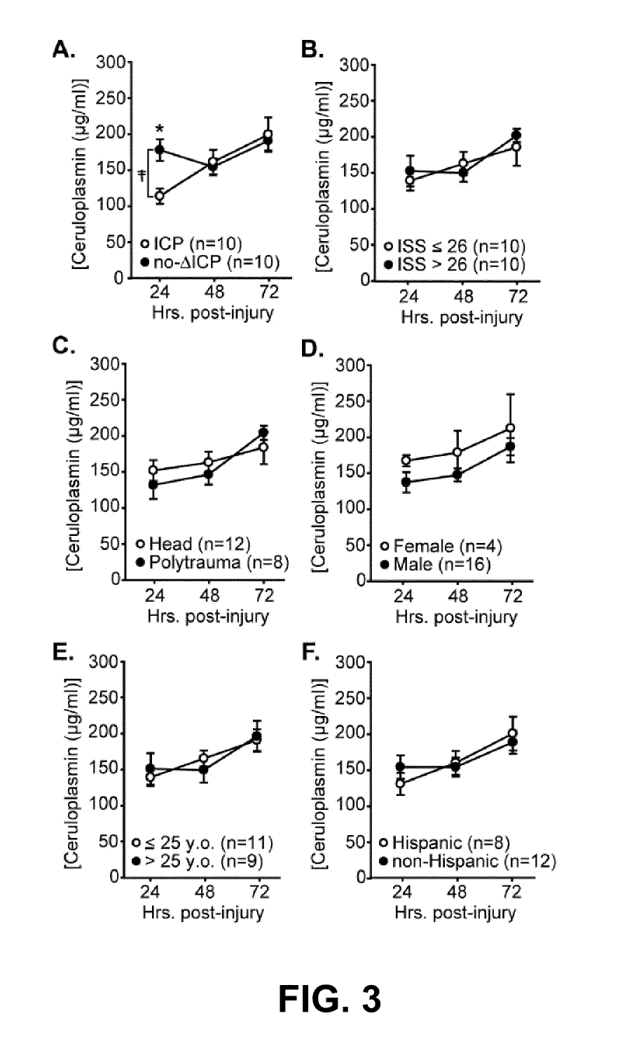

Serum samples obtained at different times following injury were analyzed by ELISA and it was determined that serum ceruloplasmin levels were differentially altered in TBI patients with increased ICP versus patients with unchanged ICP. Patients whose ICP was recorded at ≧25 mm Hg at any time during the five day sampling period were considered to have elevated ICP. The unchanged ICP group had ICP values that remained below 20 mm Hg at all sampling times. FIG. 3A shows that when classified by their ICP status, patients who experienced an elevated ICP (≧25 mm Hg) had a transient, but significant decrease in ceruloplasmin levels within the first 24 hr of injury as compared to patients whose ICP remained less than 20 mm Hg ( [symbol] , interaction by two-way ANOVA: F_{(2,51)}=3.50, p=0.038). This difference was not observed when the data was subdivided by injury severity score (F_{(2,51)}=0.446, p=0.643; FIG. 3B), by the presence of polytrauma (F_{(2,51)}=0.86, p=0.429; FIG. 3C), by gender (F_{(2,51)}=0.01, p=0.989; FIG. 3D), by age (F_{(2,51)}=0.369, p=0.693; FIG. 3E), or by ethnicity (F_{(2,51)}=0.61, p=0.547; FIG. 3F).

### Early Decline in Ceruloplasmin Levels Predicts ICP Status.

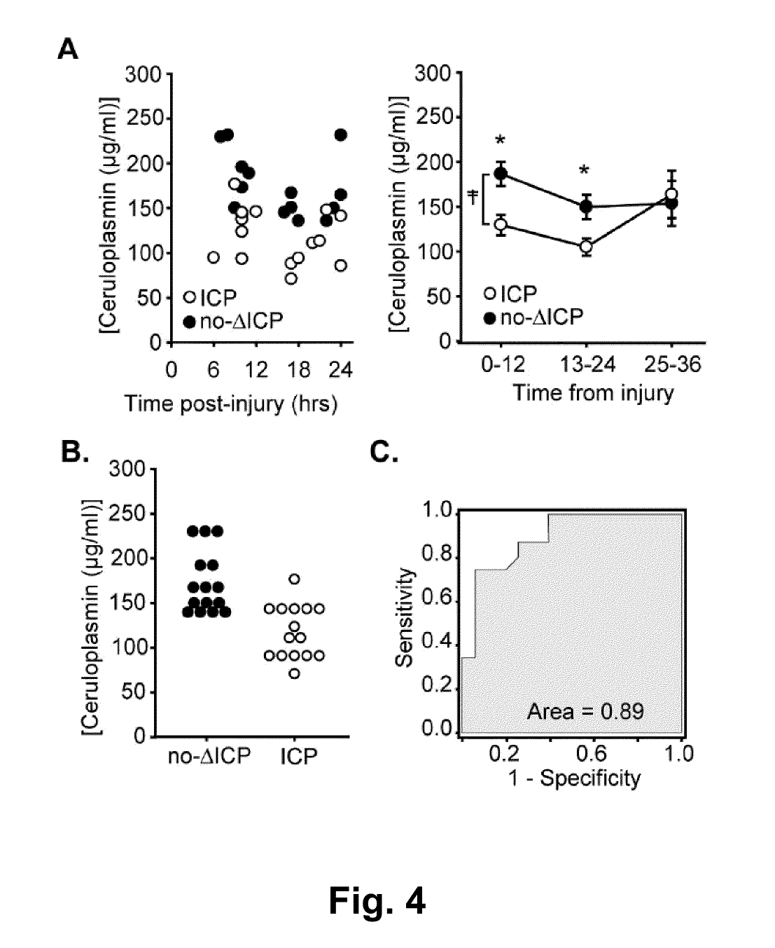

Based on the differences in ceruloplasmin levels between ICP and no-ICP groups presented in the preceding example (Example 1.5) and FIG. 3, the diagnostic value of decreases in serum ceruloplasmin levels in predicting subsequent increases in ICP in TBI patients was established by analyzing ceruloplasmin levels in serum samples taken within the first 24 hr from 15 additional subjects. FIG. 4A shows a scatter plot of individual ceruloplasmin levels as a function of sample collection time, and grouped into 12 hr bins during the first 36 hr post-injury. The figure shows that as early as the first 12 hr post-injury, a significant decrease in ceruloplasmin levels can be detected in the ICP≧25 mm Hg group ( [symbol] , group main effect by two-way ANOVA: F_{(1,2)}=4.96, p=0.033). This decrease persisted for up to 24 hr, but by 36 hr post-injury both groups had similar levels of ceruloplasmin. This data was used to generate a receiver operating characteristic (ROC) curve using the ceruloplasmin levels detected within the first 24 hr of injury (median time of 17 hrs post-injury). The concentration of ceruloplasmin in individual patients during the first 24 hr of injury is shown in FIG. 4B, subdivided into either ICP≧25 mm Hg or ICP<20 mm Hg. The area under the curve (AUC) was calculated to be 0.89, indicating that the initial serum level of ceruloplasmin is a very good predictor of subsequent rise in ICP (FIG. 4C). Using a cut-off value of 140 μg/ml, ceruloplasmin levels were determined to have a sensitivity of 87% with a specificity of 73%.

### 1.7 Serum Copper Levels Also Predict ICP Status

Since ceruloplasmin is the major copper carrier in the plasma, it was determined whether the observed decrease in ceruloplasmin levels was associated with a corresponding decrease in serum copper levels. Total serum copper levels were determined using a colormetric assay similar to that described by Abe, et al., 1989 (Abe, A., Yamashita, S. and Noma, A. 1989. Sensitive, direct colorimetric assay for copper in serum. Clin. Chem. 35, 552-554). 4-(3,5-Dibromo-2-pyridylazo)-N-ethyl-N-sulfopropylaniline, monohydrate (DiBr-PAESA) was obtained from the Dojindo Laboratory in Kumamoto, Japan. The DiBr-PAESA stock solution was prepared by dissolving 1 mg of DiBr-PAESA in 100 mL acetate buffer (0.2 mol/L, pH 5.0) containing 15 g/L SDS. The working copper color reagent was prepared by adding 1 mL of 0.35 mol/L ascorbic acid solution to 14 mL of DiBr-PAESA stock solution. To 10 μl of serum, standard, or distilled water (as a blank), 0.15 mL of the working copper color reagent was added. Following a 5 min incubation at 37° C., absorbance was measured at 580 nm. Copper concentrations were calculated from a calibration curve generated using CuSO₄ as the standard.

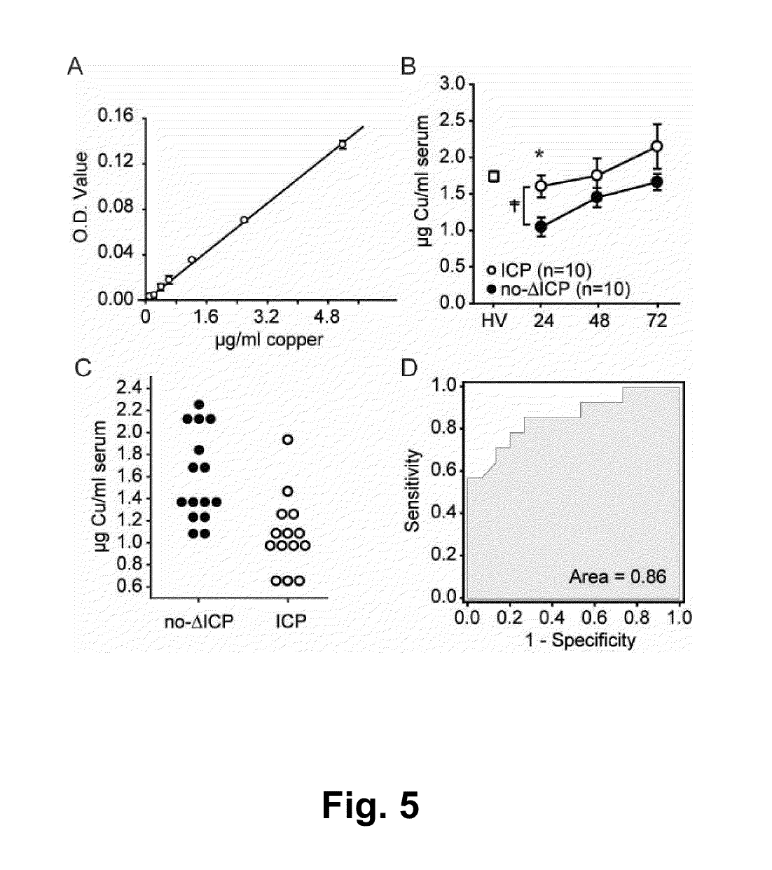

FIG. 5A shows a representative standard curve using copper sulfate as the standard. The assay was found to be linear up to the highest standard employed (5.09 μg/ml copper). When the serum samples obtained from TBI patients with ICP<20 mm Hg, and TBI patients ≧25 mm Hg were assayed for their total copper content, a significant decrease in the copper content of those TBI patients with elevated ICP was detected ( [symbol] , group main effect by two-way ANOVA: F_{(1,2)}=9.71; p=0.003). The level of total serum copper detected in healthy volunteers is shown for reference (mean±SD: 1.74±0.31 μg/ml). The distribution of serum copper levels from individual patients during the first 24 hr of injury is shown in FIG. 5C. Using these values, a ROC curve was generated that had an area under the curve measurement of 0.86, a value similar to that obtained using ceruloplasmin levels during this time frame. At a cut-off of 1.32 μg/ml copper, the data shows that the use of serum copper levels has a sensitivity of 86% and a specificity of 73% at identifying those patients who will develop a subsequent ICP elevation.

### 1.8 Combination of Ceruloplasmin and Copper Assays

The combination of ceruloplasmin and total copper were tested to determine if it would improve the diagnostic accuracy for ICP. Using a simple two-positive rule, it was found that combining serum ceruloplasmin<140 μg/ml and total serum copper<1.50 μg/ml resulted in a sensitivity of 67% (10/15 elevated ICP patients) with a 100% specificity (0/15 no-change ICP patients). Thus, both serum ceruloplasmin and total copper have prognostic accuracy in identifying patients who will develop subsequent ICP elevations. Although ceruloplasmin is readily measured using antibody-based approaches, these techniques are relatively time-consuming and may not be practical for some applications (e.g., some clinical use situations). Copper measurements, by comparison, are rapid and sensitive and may be an attractive alternate, in some cases, to measuring ceruloplasmin for the classification of patients following brain injury. As described above, the initial decreases in serum ceruloplasmin or copper were each found to have high diagnostic potential, however, they had modest specificity at the cut-off values employed (73%). In applications in which higher specificity is desired, the combination of these biomarkers can be used to minimize false positives, while maintaining relatively high sensitivity (100% specificity, 67% sensitivity). These studies demonstrate that ceruloplasmin and copper, individually and in combination, are useful as prognostic markers for development of high ICP.

### 1.9 Administration of Penicillamine does not Reduce Blood-Brain Permeability or Cerebral Edema

A number of studies have reported that blood-brain barrier compromise and cerebral edema are key contributors to the elevation of ICP. Penicillamine is a copper scavenger that is FDA approved for the treatment of Wilson's disease. Therefore penicillamine was used in this study to determine if sequestration of copper would restore TBI-associated BBB permeability and cerebral edema. In order to determine if the decrease in ceruloplasmin observed was a contributor to TBI-associated breakdown of the blood-brain barrier (BBB) and cerebral edema an established rat model of controlled cortical impact model was used to cause brain injury as described previously (Dash, P. K., Moore, A. N., and Dixon, C. E. 1995. Spatial memory deficits, increased phosphorylation of the transcription factor CREB, and induction of the AP-1 complex following experimental brain injury. J Neurosci 15, 2030-2039; Dixon, C. E., Clifton, G. L., Lighthall, J. W., Yaghmai, A. A., and Hayes, R. L. 1991. A controlled cortical impact model of traumatic brain injury in the rat. J Neurosci Methods 39, 253-262). BBB permeability was assessed by measuring the extravasation of Evans Blue dye as described in Zhao, et al., 2007 (Zhao, J., Moore, A. N., Redell, J. B. and Dash, P. K. 2007. Enhancing expression of Nrf2-driven genes protects the blood brain barrier after brain injury. J. Neurosci. 27, 10240-10248).

Cerebral edema was assessed by determining the percentage brain water content using the wet-dry method as described previously (McIntosh, T. K., Soares, H., Thomas, M. and Cloherty, K. 1990. Development of regional cerebral oedema after lateral fluid-percussion brain injury in the rat. Acta Neurochir. Suppl (Wien.) 51, 263-264; Shohami, E., Novikov, M. and Mechoulam, R. 1993. A nonpsychotropic cannabinoid, HU-211, has cerebroprotective effects after closed head injury in the rat. J. Neurotrauma 10, 109-119). BBB permeability and cerebral edema were measured 24 hr after injury, in independent sets of animals. There was no significant difference between treated and control animals indicating that penicillamine (400 mg/kg) administration 30 minutes after cortical impact injury did not have any effect on BBB permeability or cerebral edema 24 hr after the injury.

### 2.0 Treating a Head Injury Patient

An example of conventional triage and interim steps that are taken (i.e., before neurosurgery or other medical intervention) with respect to an individual with moderate to severe head injuries includes the following.

Stage I (Pre-hospital): field/transport management, initial assessment, resuscitation, stabilization and rapid transport to a trauma center (or other emergency department if trauma center not available). The patient's airway, breathing and circulation (ABCs) are the first priority as in any trauma/emergency. The patient's level of consciousness/neurological evaluation is incorporated. In head injury cases, the emergency personnel also work to avoid hypoxia and hypotension, to maintain adequate cerebral blood flow. Efforts are made to ensure that the patient has adequate circulating oxygen (e.g., making sure that the airway is clear, assessing whether intubation is necessary, providing supplemental oxygen, monitoring blood oxygenation, for example, using a pulse dosimeter checking blood gas levels) and maintaining the blood pressure (e.g., systolic pressure above 90 mm Hg to maintain a cerebral perfusion pressure (CPP) of at least 60 mm Hg. Intravenous fluids may be given and treatment to stop bleeding is provided. Glasgow Coma Score (GCS), pupil response and size and vital signs are assessed both in the field, on admission to the emergency department and routinely thereafter.

A patient who cannot protect his airway, typically a GCS<8 will be intubated to assure adequate oxygenation. Intubation may occur in the field or in the hospital. One of the confounders in treating head injury patients is that in order to intubate a patient, medications such as paralytics, neuromuscular blockers and sedatives are administered, which can affect the patient's neurological status. Additionally, in some instances a patient has other substances such as alcohol and drugs in his body that can also affect his neurological status. It may take a significant amount of time for these medications to wear off. Both the head and neck of the patient are immobilized until the spine can be examined and concomitant spinal cord injury ruled out.

Stage II (Hospital or Trauma Center): Stabilization and resuscitation efforts continue. If available, a computed tomography (CT) scan of the patient's head is performed. If the patient has substances in his body that can influence his neurological status, and/or the patient's CT scan result is inconclusive, the patient may be observed for a period of 6 to 12 hours prior to placing an ICP monitor/ventriculostomy and repeating the head CT.

Mannitol and/or hypertonic saline may be given to lower intracranial pressure if the patient has received adequate fluid volume. In cases where true evidence of elevated ICP or impending herniation exists, hyperventilation can be used for a short term. As there is a rebound effect from hyperventilation treatment, this is usually suggested only for emergent management. Sedation, analgesics and paralytics may also be used for suspected elevated ICP.

In addition to the above-described emergency treatment steps, emergency department work up includes laboratory studies including blood gases, complete blood count, electrolytes, coagulation, toxicology (not always performed), radiographs including chest x-ray, head CT, spine CT, as well as the abdomen and other organs if other injuries suspected.

Stage III (Neurosurgical): Neurosurgery personnel are typically consulted for positive CT scans or for patients with an altered level of consciousness. If a patient has an operative lesion on CT they are transferred to the operating room emergently. An ICP monitor and/or ventriculostomy is placed for a patient requiring ICP monitoring/drainage for elevated pressure, typically those with GCS<8. Neurosurgery personnel are involved at this point, although the emergency department could place a monitor.

Medications to prevent seizures are typically started and given as prophylactic treatment for 7 days. The patient's temperature is controlled to prevent the patient from becoming hyperthermic, which causes an increased metabolic rate and increased oxygen demand. Refractory elevated ICP typically goes through the treatment continuum of sedation, mannitol, CSF drainage, paralytics, barbiturate coma, and decompressive craniectomy.

A conventional head injury treatment regime is improved by obtaining advance detection of impending elevation of ICP in the patient, as described above. This improvement is made possible by the discovery that in at-risk patients, those who go on to develop elevated ICP have a significant but transient reduction in serum ceruloplasmin and serum copper levels below the normal range shortly after injury but well before elevated ICP occurs or peaks. For example, the level of serum ceruloplasmin and/or copper is determined within the first 24 hrs after the injury or other precipitating event. In some embodiments, the patient's serum is tested for ceruloplasmin and/or copper levels. In some cases, levels of copper and/or ceruloplasmin in a body fluid other than serum (e.g., plasma, blood, cerebral spinal fluid, urine and saliva) are used as surrogate markers indicative of serum levels.

By screening the head injury patient for an early post-injury drop in ceruloplasmin and/or copper levels while the patient is still in Stage I or Stage II care (as described above), it can be better predicted which of those individuals will go on to develop elevated ICP. This knowledge provides an early warning system that facilitates the more rapid and appropriate deployment of resources, allowing time to transport a patient from the field to a tertiary care center, or allows time to get a neurosurgeon and surgical team in place before the onset of dangerous ICP. In this way, the patient has a better chance of reducing or avoiding brain damage from elevated ICP.

### 3. Detection of Elevated ICP Risk in Veterinary Animals

In addition to diagnosing and/or treating a population of human at-risk patients, and identify individuals that will go on to develop elevated ICP, those of skill in the art will readily recognize that the disclosed methods also extend to animals and that early indication of elevated ICP will be as useful to veterinarians as it is to physicians. In fact, given the rural nature of many veterinary practices and the far more limited availability of technically advanced veterinary medical centers, the process of the present invention may prove even more useful to those in veterinary practice. For example, racing horses and dogs are very valuable and yet they are often subject to race or training related trauma and possibly a life threatening elevation in ICP. Likewise, companion animals (dogs and cats) often travel in motor vehicles with their owners, unsecured and are therefore at risk of TBI resulting from a motor vehicle accident. Similarly, companion animals live in close proximity to motor vehicles and are therefore at risk of being struck and suffering associated brain injuries and accompanying deleterious elevation in ICP. Valuable large animals such as horses and cattle may also suffer brain injury, often far from medical care. For some applications, a particularly useful embodiment comprises a test kit for field use that could be used by a local veterinarian, animal trainer or other individual, as a way of identifying animals that will develop elevated ICP.

In addition to veterinary copper assays which are already available for other purposes, assays used to determine copper levels in the body fluids of humans could also be used to determine copper levels in the body fluids of non-human mammals. Given the homology between human ceruloplasmin and that of other mammals, it is likely that assays that measure human ceruloplasmin will also be useful for measuring ceruloplasmin in other mammals. For example, a particular human ceruloplasmin antibody in an immunoassay might cross react with that of other mammals. Alternatively, the generation of a cross reactive polyclonal or monoclonal antibody that binds to both human and non-human ceruloplasmin would be fairly easy to obtain. Alternatively, should one desire ceruloplasmin antibodies specific to a target species, such antibody may be generated, identified and characterized using the methods described herein.

### Test Kits

In some embodiments, identifying a patient at risk for developing elevated ICP includes determining the amount of ceruloplasmin and/or copper present within a biological sample from the patient, wherein the presence of decreased ceruloplasmin and/or copper in comparison to a sample from a normal subject, is indicative of a patient developing elevated ICP. All the essential materials and reagents required for conducting such determinations may be assembled together in a kit to facilitate the rapid and easy identification of patient as being at risk for elevated ICP. With the kit, the user determines whether a particular sample of a patient's body fluid contains or indicates a level of serum ceruloplasmin that is below the normal range, and may also determine whether a particular sample of a patients body fluid contains or indicates a level of serum copper that is below the normal range. These assays may be performed as two distinct assays, using, for example, two test strips. One test strip would contain an immunoassay technology for assaying ceruloplasmin and the other test strip would provide a colorimetric test for copper. Alternatively, in some embodiments both tests are performed using a single test strip that contains both a detection system for ceruloplasmin and a detection system for copper.

For example, in some embodiments an immunoassay detection kit is provided that comprises at least a first antibody (e.g., a monoclonal antibody) capable of binding to a ceruloplasmin protein or peptide and a detection reagent, along with testing instructions. In some instances, the immunoassay detection kit also includes an unrelated protein or peptide for use as a control, and/or a second antibody that binds to the first antibody. In some embodiments, the kit contains the necessary components for testing a body fluid for copper level, to determine whether the individual is at increased risk of elevated ICP.

As any serum copper level reduction will be detected in a brain injured individual's body fluid within 24 hrs., in many applications a copper dipstick test will be preferred. Other potential advantages of copper test kits are due to copper being more stable than ceruloplasmin, and copper colorimetric assays are readily available. Some test kits may contain a bimodal dipstick test for measuring both copper and ceruloplasmin.

A test kit may have a single container or it may include individual containers for each reagent. When test components are provided in the form of one or more liquid solutions, they are preferably provided as a sterile aqueous solutions. Reagents may also be provided in dried or lyophilized forms. When reagents or components are provided as a dried form, reconstitution generally is by the addition of a suitable solvent. The solvent may be provided in another container means. The kit may also include one or more vials, test tubes, flasks, bottles, syringes or other suitable containers, into which the test reagent formulation is placed, preferably suitably allocated. In some embodiments, a kit also comprises a second container for containing a sterile, pharmaceutically acceptable buffer or other diluent. For example, the container may itself be a syringe, pipette, or other dispensing device that can be used for applying or mixing with other components of the kit. Irrespective of the number or type of containers employed in a kit, in some embodiments the kit also includes, or is packaged with, an instrument for signal detection and analysis. In some embodiments, a kit includes suitable packaging for holding the various components (e.g., vials) in close confinement for commercial sale such as, for example, an injection or blow-molded plastic container in which the desired vials are retained. Instructions for use of the kit components may be provided in the kit.

### Exemplary Embodiments

In certain embodiments, a rapid, inexpensive and effective method is provided that identifies individuals at increased risk of developing elevated ICP. In certain embodiments, a process for identifying a patient at risk for elevated intracranial pressure comprising analyzing a test sample obtained from the patient for ceruloplasmin levels, copper levels or both and determining that serum ceruloplasmin levels, copper levels or both levels are decreased below the normal range on a patient that has experienced a possible brain injury. In certain embodiments the test sample comprises a patient's serum. In some other embodiments the test sample comprises a body fluid comprising plasma, blood, urine, cerebral spinal fluid, urine or saliva. In some cases, levels of copper and/or ceruloplasmin in a body fluid other than serum are used as surrogate markers indicative of serum levels. In various applications, the patient's possible brain injury is due to traumatic brain injury, head trauma, polytrauma, stroke, meningitis, encephalitis, hydrocephalus, spinal cord injury, blocked shunts, or a tumor of the central nervous system.

In certain embodiments, a process for identifying a patient at risk for elevated intracranial pressure comprising analyzing a test sample obtained from the patient for ceruloplasmin levels and determining that ceruloplasmin levels are decreased below the normal range on a patient that has experienced a possible brain injury.

In certain embodiments, the process comprises the use of an immunoassay to determine if a patient's serum ceruloplasmin level is below the normal range. In certain embodiments the immunoassay comprises an enzyme linked immunoassay, or a radioimmunoassay, or immunonephelometry, or immunochromatography, or a lateral flow test strip, and any other immunoassay capable of detecting ceruloplasmin in a biological sample and correlated to serum levels.

In certain embodiments, a process for identifying a patient at risk for elevated intracranial pressure comprising analyzing a test sample obtained from the patient for copper levels and determining that serum copper levels are decreased below the normal range in a patient who has experienced a possible brain injury. In certain embodiments the analysis of the copper levels comprises colorimetry, of atomic absorption, or spectrophotometry.

In certain embodiments, a process for identifying a patient at risk for elevated intracranial pressure comprises analyzing a test sample obtained from the patient for ceruloplasmin levels and determining that serum ceruloplasmin levels are decreased below the normal range on a patient that has experienced a possible brain injury.

In certain embodiments, a method of identifying an individual as being a candidate for a invasive intracranial pressure monitoring technique the method comprising, analyzing a test sample of body fluid obtained from the individual after a the possible brain injury for the ceruloplasmin level, the copper level, or both, in the sample and determining that the serum ceruloplasmin level, the serum copper level, or both, are decreased below the normal range.

In certain embodiments, a method of identifying a potential brain injury patient as being a candidate for a invasive intracranial monitoring techniques, the method comprising analyzing a test sample from the patient for ceruloplasmin, copper, or both, and determining that the patient's serum contains ceruloplasmin and copper levels that are decreased below the normal range.

In certain embodiments, a process of identifying an individual as being a candidate for invasive intracranial monitoring techniques, the process comprising, analyzing a test sample obtained from the individual within 36 hours of a possible brain injury for the ceruloplasmin level, the copper level or both in the test sample and determining that the serum ceruloplasmin level, the serum copper level, or both, are decreased below the normal range. In certain embodiments, wherein the invasive intracranial pressure monitoring technique comprises the use of a bolt (e.g., a Camino bolt)) or a ventriculostomy. In certain embodiments, a process for transforming the status of a patient from being theoretically at risk for elevated intracranial pressure to being at actual risk for elevated intracranial pressure, the process comprising, analyzing a test sample obtained from the patient for ceruloplasmin levels, copper levels, or both, and determining that the patient's serum ceruloplasmin levels, serum copper levels, or both, are decreased below the normal range on a patient that has experienced a possible brain injury event.

In certain embodiments, a process of identifying an individual who has experienced possible brain trauma as being at high risk for dangerously elevated intracranial pressure, the process comprising: analyzing a test sample obtained from the individual for the ceruloplasmin level, the copper level or both in the sample and determining that the serum ceruloplasmin level, the serum copper level, or both, are decreased below the normal range, and thus identifying the individual as being a valid candidate for more invasive monitoring techniques and closer observation. In certain embodiments, the patient or individual is a human, a dog, a cat, a horse or other mammal.

In some embodiments, an above-described test kit comprises materials for performing an immunoassay for ceruloplasmin. In some embodiments, an above-described test kit comprises materials for performing a colorimetric test for copper, which in some cases includes a dipstick for testing a body fluid of the individual. In some embodiments, a first set of components in a test kit disclosed herein includes materials for performing a colorimetric test for copper. In certain embodiments in which the body fluid tested is not serum (e.g., plasma, blood, urine, cerebral spinal fluid, urine or saliva), a test kit includes literature that also contains instructions for correlating a measured copper level in a body fluid other than serum to a serum copper level. In some embodiments, the literature included in a test kit includes instructions for correlating a measured ceruloplasmin level in a body fluid other than serum to a serum ceruloplasmin level. In some embodiments, a second set of components comprises materials for performing an immunoassay for ceruloplasmin. For example, in some cases components for performing a ceruloplasmin immunochromatography assay using a lateral flow test strip are included, and in some cases the materials in an above-described kit includes a dipstick test for dipping into a test sample of a body fluid.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A process for identifying a patient at risk for elevated intracranial pressure, said patient having experienced a precipitating event that includes brain injury or potential brain injury, the process comprising:
measuring a ceruloplasmin level, copper level, or both ceruloplasmin and copper levels in a test sample of body fluid obtained from the patient, wherein said test sample is obtained within 36 hours after the precipitating event; and
determining from said measurement that the serum ceruloplasmin level, copper level, or both levels, are below the respective normal ranges, wherein a subnormal level of either serum ceruloplasmin or serum copper, or both, indicates increased risk for elevated intracranial pressure.

2. The process of claim 1, wherein the body fluid comprises serum.

3. The process of claim 1, wherein the body fluid is selected from the group consisting of serum, plasma, blood, urine, cerebral spinal fluid, urine and saliva, and said determining comprises correlating said measured levels to a serum ceruloplasmin level, a serum copper level, or to both levels.

4. (canceled)

5. The process of claim 1, wherein said measuring comprises measuring ceruloplasmin level.

6. The process of claim 5, wherein said measuring comprises performing an immunoassay.

7.-8. (canceled)

9. The process of claim 1, wherein said measuring comprises measuring a copper level.

10.-12. (canceled)

13. The process of claim 1 further comprising:
identifying said patient as a candidate for more invasive monitoring techniques and closer observation, based on said determination that the serum ceruloplasmin level, the serum copper level or both levels are below the respective normal ranges; and
using an invasive intracranial pressure monitoring technique on the patient.

14. (canceled)

15. The process of claim 1 further comprising:
after said precipitating event and prior to said measuring and determining, establishing a first diagnosis of the patient as being theoretically at risk for elevated intracranial pressure; and
after said measuring and determining, establishing a second diagnosis of the patient as being at increased risk for elevated intracranial pressure, based on said determination that the serum ceruloplasmin level, serum copper level or both levels are below the respective normal ranges.

16.-17. (canceled)

18. The method of claim 1, wherein said patient is included in a population of individuals who have experienced a precipitating event that may include or cause brain injury, the method comprising:
measuring a ceruloplasmin level, copper level, or both ceruloplasmin and copper levels in said test sample obtained from each said individual, wherein each said test sample is obtained within 36 hours after the respective precipitating event;
determining from each said measurement whether the serum ceruloplasmin level, serum copper level or both levels are below the respective normal ranges, wherein a subnormal level of either serum ceruloplasmin or serum copper, or both, indicates increased risk for elevated intracranial pressure; and
monitoring at increased frequency and level of intensity for neurological changes at least the individuals whose said determinations indicate increased risk of elevated intracranial pressure.

19. A method of treating an individual who has experienced a precipitating event that includes or may cause brain injury, the method comprising:
performing the process of claim 1; and
monitoring said patient at an increased frequency and level of intensity for neurological changes.

20.-21. (canceled)

22.-23. (canceled)

24. The method of claim 19, further comprising
administering to said patient a treatment to deter or alleviate elevated intracranial pressure.

25.-42. (canceled)

43. The process of claim 1, wherein said measuring comprises measuring said copper level, and said sample is obtained within 24 hours after said precipitating event.

44. (canceled)

45. A test kit for identifying an individual at increased risk of elevated intracranial pressure, comprising:
(a) a first set of components for measuring a copper level in a body fluid sample according to the process of claim 1; and
(b) literature containing instructions
(i) for obtaining said sample from said individual within 36 hours after a precipitating event that includes brain injury or potential brain injury, in said individual;
(ii) for using said first set of components to measure said copper level in said body fluid-sample according to the process of claim 1; and
(iii) for interpreting a copper level measurement according to the process of claim 1, wherein a subnormal level of serum copper indicates increased risk of elevated intracranial pressure.

46. The test kit of claim 45, further comprising:
(c) a second set of components for measuring ceruloplasmin levels in a second body fluid sample,
wherein said literature further comprises instructions
(iv) for using said second set of components to measure ceruloplasmin level in said second body fluid sample according to the process of claim 1; and
(v) for interpreting a ceruloplasmin level measurement according to the process of claim 1, wherein a subnormal level of serum ceruloplasmin indicates increased risk of elevated intracranial pressure.

47. The test kit of claim 45, wherein said first set of components comprises materials for performing a colorimetric test for copper.

48. The test kit of claim 45, wherein said literature further comprises instructions for correlating a measured copper level in a body fluid other than serum to a serum copper level.

49. The test kit of claim 48, wherein said literature further comprises instructions for correlating a measured ceruloplasmin level in a body fluid other than serum to a serum ceruloplasmin level.

50. The test kit of claim 46, wherein said second set of components comprises materials for performing an immunoassay for ceruloplasmin.

51. The test kit of claim 50 wherein said second set of components comprises materials for performing a ceruloplasmin immunochromatography assay using a lateral flow test strip.

52. The test kit of claim 45 wherein said first set of components comprises a dipstick for dipping into said body fluid sample.
